(19) United States
(12) Patent Application Publication
(10) Pub. No.: US 2026/0201313 A1
(43) Pub. Date: Jul. 16, 2026

(54) USE OF MICROALGAE AND MICROALGAE DERIVED COMPONENTS IN CULTIVATED FOOD PRODUCTION

(71) Applicant: WEFORESEA LTD., Ness-Ziona (IL)

(72) Inventors: Rotem KADIR, Rosh Haayin (IL); Tomer HALEVY, Nahariyya (IL)

(73) Assignee: WEFORESEA LTD., Ness-Ziona (IL)

(21) Appl. No.: 19/136,195

(22) PCT Filed: Dec. 22, 2023
(86) PCT No.: PCT/IB2023/063208

§ 371 Date: Jun. 5, 2025

Related U.S. Application Data
(60) Provisional application No. 63/435,400, filed on Dec. 27, 2022.
(60) Provisional application No. 63/435,434, filed on Dec. 27, 2022.

(51) Int. Cl. C12N 1/12; A23L 17/00; C12N 1/02; C12N 5/071

(52) U.S. Cl. CPC C12N 1/12; A23L 17/00; C12N 1/02; C12N 5/0602

(57) ABSTRACT

Provided herein are methods for cultivated food production involving the use of microalgae. The methods include recycling spent cell media that are used to cultivate cells for cultivated food production as well as enriching culture medium used to cultivate the same cells. The microalgae are used to recycle the spent culture medium. The microalgae may be used to extract microalgae derived components to enrich the culture medium. The algae recycling and algae enriched growth media as provided herein may be used with any animal cell culture. In some variations, the microalgae or microalgae derived components may be used as a raw ingredient in the preparation of cell based seafood products. The methods provided herein may be used for the production of seafood products. The methods herein may provide at least one added advantage—the use of algae may add nutrients, flavors, and aromas typically desired in seafood.

### CROSS-REFERENCE TO RELATED APPLICATIONS

This application claims priority to and the benefit of U.S. Provisional Application No. 63/435,400, filed on Dec. 27, 2022 and U.S. Provisional Application No. 63/435,434, filed on Dec. 27, 2022, each of which is incorporated herein by reference in its entirety.

### FIELD

The present disclosure relates generally to cell-based food products, and more specifically to methods and systems for treating and recycling of cells spent media and enriching the growth media with algae-based components in the production of such cell-based food products.

### BACKGROUND

Animal cells exploit nutrients from culture media, additionally, they excrete ammonia and other secondary metabolites that are harmful to cells, slow down cell division, and may even cause cell death in high concentrations. Generally, the waste medium is discarded and replaced with a new culture medium because of reduced nutrients and the accumulation of waste products. Large volumes of discarded waste medium can lead to environmental problems. Therefore, there is a growing need for the development of commercially feasible technologies for the treatment and recycling of cells spent media.

### BRIEF SUMMARY

In various aspects, provided herein are methods for culture medium recycling, suitable for use in cultivated food production. In some embodiments, the algae recycling and algae enriched growth media as provided herein may be used with any animal cell culture. The methods herein provide a commercially viable approach for use in cultivated food production that reduces cost, and enhances culture performance. In some variations, the methods provided herein may be used for the production of seafood products. In such variations, the methods herein provide at least one added advantage—the use of algae may add nutrients, flavors, and aromas typically desired in seafood.

In some aspects, provided is a method for cultivated food production, comprising: (i) cultivating cells in a media, optionally wherein the media comprises enriched media; (ii) withdrawing spent media resulting from the cultivating of cells in step (i); (iii) proliferating algae in the presence of ammonia and/or ammonium from the spent media; (iv) lysating the algae; and (v) retrieving treated spent media to further cultivate cells in step (a). In some variations, at least a portion of the cultivated cells in step (i) are harvested at any time.

In some aspects, provided is a method for cultivated food production, comprising: (a) cultivating cells in a media, optionally wherein the media comprises enriched media; (b) recycling spent media resulting from the cultivating of cells in step (a), wherein the recycling of the spent media is performed by microalgae; and (c1) expanding the cells until a biomass of cultivated cells is reached, or (c2) harvesting at least a portion of the cells and (d2) allowing remaining cells to expand upon repeating steps (a)-(c1) above. In some embodiments of the foregoing, the media is enriched by microalgae derived components.

In one aspect, provided herein is a method for cultivated food production, comprising: (a) cultivating cells in enriched media; (b) recycling spent media resulting from the cultivating of cells in step (a), wherein the recycling of the spent media is performed by microalgae; and (c) expanding the cells until a biomass of cultivated cells is reached.

In some aspects, provided herein is a method for cultivated food production, comprising: (a) cultivating cells in enriched media; (b) recycling spent media resulting from the cultivating of cells in step (a), wherein the recycling of the spent media is performed by microalgae; (c) harvesting at least a portion of the cells; and (d) allowing remaining cells to expand upon repeating steps (a)-(c1) above. In other words, in some variations, the methods provided herein may be a continuous process that involves harvesting some of the cell mass, and letting that cell mass expand again repeatedly for an extended time period, e.g., several months. During this time, algae treated media is constantly reintroduced to the animal cell culture.

In other aspects, provided herein is a method for cultivated food production, comprising: (a) cultivating cells in enriched media, wherein the media is enriched by microalgae derived components; (b) recycling spent media resulting from the cultivating of cells in step (a), wherein the recycling of the spent media is performed by microalgae; and (c) expanding the cells until a biomass of cultivated cells is reached.

In certain aspects, provided herein is a method of recycling spent media from the cultivation of cells for cultivated food production, comprising: (a) separating the spent media from the cultured cells; (b) introducing microalgae to the spent media; (c) cultivating the microalgae under heterotrophic, autotrophic, and/or mixotrophic conditions, e.g., to recycle the spent media, reducing the concentration of ammonia; and (d) separating the microalgae from the recycled media. In some variations of the foregoing aspect, the microalgae is cultivated under heterotrophic conditions.

In yet other aspects, provided is a method of extracting microalgae derived components, comprising: (a) lysing microalgae cells, wherein the lysing is performed by high pressure homogenization; (b) treating the lysed microalgae cells with a detergent to release soluble proteins; (c) centrifugating the lysed microalgae cells treated with the detergent; (d) collecting the supernatant; and (e) concentrating the extracted microalgae derived components from the supernatant by centrifugation.

In some aspects, provided is a method comprising: a) cultivating cells with suitable medium components; b) withdrawing spent media; c) adding the withdrawn spent media from step b) to a container comprising microalgae; c) proliferating algae in the container in the presence of ammonia and/or ammonium; d) lysating the algae; and e) retrieving treated spent media to additional cell culture, with or without algae lysate and/or additional suitable medium components. In the foregoing aspects, in step c), the presence of the ammonia and/or ammonium comes from the spent media. In other words, the ammonia and/or ammonium used does not come from other external sources. In some variations, the amount of ammonia and/or ammonium present may vary. In some embodiments, the spent media may be supplemented with glucose to help the algae metabolize the ammonia and/or ammonium. In some embodiments, the algae-treated spent media may be supplemented with algae lysate and/or suitable medium comments (such as vitamins, amino acids, and growth factors). The cultivating of the cells, recycling of spent media, and lysating of algae may be performed in accordance with the various embodiments described herein. Further, in the foregoing aspect, in some embodiments, steps a)-e) may be repeated one or numerous times. Further, in other embodiments, the harvesting may be performed in various ways in any phase of the cycle. In some variations, the methods described herein may be used for any suitable cell-lines.

In some aspects, provided herein is an extract containing microalgae derived components produced by the methods described herein. Also provided herein is an enriched culture media enriched by the extract. Additionally provided is an enriched culture media produced by the methods described herein.

In other aspects, provided herein is a biomass of cultured cells produced by the methods described herein. Also provided is a biomass of cultured cells produced by the methods described herein and cultivated with the enriched culture medium produced by any of the methods described herein.

In yet other aspects, provided is a cultivated seafood product produced by the methods described herein.

### DESCRIPTION OF THE FIGURES

The present application can be understood by reference to the following description taken in conjunction with the accompanying figures.

FIG. 1 depicts an exemplary process for recycling, extracting and formulating media using microalgae.

FIG. 2 depicts an exemplary process for recycling spent media and adjusting the recycled, spent media.

FIG. 3 depicts the growth of two species of microalgae on spent media that was recycled from fish cell growth.

FIGS. 4A-4B depicts ammonia (NH₃) reduction over time by microalgae for media recycling. FIG. 4A indicates ammonia reduction by growth hours with a continuous x-axis whereas FIG. 4B indicates ammonia reduction at distinct time-points. Spent media: (−) medium without algae cells (this was used as a control for testing NH3 stability in the medium); (+) medium with algae cells.

FIGS. 5A-5B depicts large-scale ammonia (NH3) utilization by microalgae for media recycling. FIG. 5A represents the initial concentration of each metabolite in the medium before the algae recycling process at day zero (DO), compared to their concentration after two days of the microalgae recycling process (D2). Ammonia (NH3) is represented in millimolar (mM); glucose (Glc) is represented in grams/L (g/L); Glutamine (GLN) is represented in millimolar (mM); and pyruvate is represented in millimolar (mM). FIG. 5B represents the percent change of metabolites after microalgae treatment, 100*(D2−D0)/D0). “NH3” is ammonia, “Glc” is glucose, “GLN” is glutamine and “Pyr” is pyruvate.

FIG. 6 depicts exemplary phosphate reduction by cultivated microalgae on spent media that was recycled from fish cell growth. FIG. 6 represents the percentage of phosphate on the first timepoint (T1—day 1) and the second timepoint (T2—day 2), both compared to the initial concentration of phosphate on day 0. Phosphate is reduced in the control media and algae recycled (ARM) media samples. Phosphate is not reduced in the negative control samples. Control-growth on algae reference media (BG11 medium). ARM-algae recycled media. BG11 (No algae) and Spent no algae are negative controls. Tl is Day 1. T2 is Day 2.

FIG. 7 depicts the exemplary process for obtaining microalgae extracts and enriching growth media with microalgae-derived components.

FIG. 8 depicts fish cells grown in different FBS concentrations supplemented with or without 0.01 mg/ml of microalgae derived extracts.

### DETAILED DESCRIPTION

The following description sets forth exemplary systems, compositions, methods, parameters and the like. It should be recognized, however, that such description is not intended as a limitation on the scope of the present disclosure but is instead provided as a description of exemplary embodiments.

Cell cultures have been established and used to study animal cell behavior in vitro since the beginning of the last century. By the end of the same century, cell culture techniques had progressed immensely to the point where it was possible to produce and isolate biological molecules to use as medicinal drugs. These advances in cell culture abilities paved the way to the establishment of different applications and fields of research such as the biosimilar drug industry, regenerative medicine cell-based therapies, vaccine and antibody production, and in recent years, the cultured meat or seafood industry, all of which rely on highly precise and controlled cell culturing processes. These advances however, focused on cellular growth, characteristics and applications while costs of production remained high. This is most notable in cellular fermentation-based processes such as those described above, where cells must be expanded to high concentrations in large volumes. The major cost component of cell mass production is the feed of the cells, also referred to as the growth media. Being based on animal components or chemically defined, media production is still made by specialized pharma companies under pharma-grade regulation and accordingly, down-stream applications based on these media products are expensive. To lower costs of production, several changes must be made, first and foremost, a more sustainable and affordable source of essential molecules must be used to serve as the core of the media. Reducing media prices will benefit both the industry and consumers, lowering biosimilar drug prices and enabling the cultured meat or seafood industry to commence scale-up production of cell-based raw material for food applications, as the current production prices are not feasible for industrialization.

In various aspects, provided herein are methods for culture medium recycling, suitable for use in cultivated food production. In some embodiments, the methods involve algae treating spent media for any animal cell culture. In some variations of the foregoing, the methods involve algae treating spent media for fish cell culture. The methods herein provide a commercially viable approach for use in cultivated food production that reduces cost, and enhances culture performance. In some variations, the methods provided herein may be used for the production of seafood products, and the use of algae may add nutrients, flavors, and aromas typically desired in seafood. The circular methods described provide a commercially viable recycling process that enables a high-performance yet low-cost bioprocess.

Further, in accordance with the methods described herein, the algae grown on spent media can also be used as a raw material to be mixed in the preparation of cell-based seafood products. In some embodiments, the algae systems as described in further detail herein may be used with any animal cell culture. In certain embodiments, the algae systems as described herein may be used to create seafood products. For example, in some variations, fish cells may be used in the methods described herein to create various types of seafood products, including scallops, shrimp and crab. Thus, in certain variations, a fish culture is a platform in accordance with the methods described herein may be used to produce a variety of seafood products.

### I. Cultivation Methods

In some aspects, provided is a method for cultivated food production, comprising: (i) cultivating cells in a media, optionally wherein the media comprises enriched media; (ii) withdrawing spent media resulting from the cultivating of cells in step (i); (iii) proliferating algae in the presence of NH₃ from the spent media; (iv) lysating the algae; and (v) retrieving treated spent media to further cultivate cells in step (a). In some variations, at least a portion of the cultivated cells in step (i) are harvested at any time.

In some aspects, provided is a method for cultivated food production, comprising: (a) cultivating cells in a media, optionally wherein the media comprises enriched media; (b) recycling spent media resulting from the cultivating of cells in step (a), wherein the recycling of the spent media is performed by microalgae; and (c1) expanding the cells until a biomass of cultivated cells is reached, or (c2) harvesting at least a portion of the cells and (d2) allowing remaining cells to expand upon repeating steps (a)-(c1) above. In some embodiments of the foregoing, the media is enriched by microalgae derived components.

In some embodiments, the method comprises culturing the cell on a growth medium comprising microalga-derived material under conditions suitable for proliferation of the cell. In some embodiments, a microalga-derived material comprises: extract, homogenate, lysate, any fraction thereof, or any combination thereof, being derived from a microalga. As used herein, the term “microalga” refers to any aquatic photosynthetic organism, being a single cell organism. In some variations, microalgae refers to (but not limited to) single-cell or groups of cells joined together from the Kingdom Protista and refers to all photosynthetic protists, such as phytoplankton, cyanobacteria, diatoms, dinoflagellates, etc.

In some embodiments, the culturing is under a temperature ranging from 10-30° C., 20-28° C., 10-20° C., 10-25° C., 14-30° C., 18-30° C., 15-25° C., 8-20° C., 10-42° C., 18-40° C., 20-39° C., or 17-28° C. Each possibility represents a separate embodiment of the invention.

In some embodiments, the culturing is under pH ranging from 6-8, 7-8, 7.1-8, 7.2-8, 7.3-8, 7.4-8, 7.5-7.9, 7.6-7.9, or 7.6-7.8. Each possibility represents a separate embodiment of the invention.

In some embodiments, the culturing is under CO₂ supplementation. In some embodiments, CO₂ supplementation is 5% CO₂ supplementation, as would be apparent to one of ordinary skill in the art.

In some aspects, provided herein are methods for cultivated food production. In some embodiments, the method comprises: (a) cultivating cells in enriched media; (b) recycling spent media resulting from the cultivating of cells in step (a), wherein the recycling of the spent media is performed by microalgae; and (c) expanding the cells until a biomass of cultivated cells is reached. In some variations, “food product” refers to a material, a substance, or an additive, which can be used as food, or which can be added to food. In some variations, the food product is any composition than an animal, preferably a mammal such as a human, may consume as part of its diet.

In certain embodiments of the foregoing method, the media is enriched by microalgae-derived components. The methods provided herein can be used for the cultivation of any type of cells for the development of food products. In certain variations of the foregoing, the method further comprises creating stable cell lines. Any suitable methods known in the art to create such stable cell lines may be employed.

In some embodiments, a cell is selected from: a primary cell culture, an immortalized cell, or a cell line. In some embodiments, a cell is derived from a muscle tissue of an aquatic organism. In some embodiments, a cell is a myoblast, a myotube, a myocyte, a cardiac muscle cell, a skeletal muscle cell, a myofiber, or any progenitor cell thereof.

In some embodiments, the cells cultivated in step (a) are from a cell line.

In some variations, the following cell lines may be used, and can be obtained from the ATCC cell bank and cultured according to the bank's protocols.
Snail Biomphalaria glabrata Say, 1818 embryonic (Bge) cell line (https://www.beiresources.org/Catalog/cellBanks/NR-40248.asox).
Chicken: SL-29 CRL-1590™ (https://www.atcc.org/products/crl-1590), DF-1 chicken embryonic fibroblasts (https://www/atcc.org/products/crl-12203).
Bovine: FBHE CRL-1395™ (https://www.atcc.org/products/crl-1395).
Pig: PK (15) CCL-33™ (https://www.atcc.org/products/crl-33).
Fish: RTgill-W1CRL-2523™ (https://www.atcc.org/products/crl-2523), RTG-P1CRL-2829™ (https://www.atcc.org/products/crl-2829) rainbow trout.
Pharma industry (hamster): CHO-KI CCL-61™ (https://www.atcc.org (products/ccl-61).
Pharma industry (human): 293 [HEK-293] CRL-1573™ (https://www.atcc.org/products/crl-1573).
Mouse: C57BL/6 SCRC-1002™ (https://www.atcc.org/products/scre-1002).
SF9 CRL-1711 (https://www.atcc.org/products/crl-1711).
EB66 (https://www.atcc.org/products/ccl-141).
DFl (https://www.atcc.org/products/crl-12203).
LMH (https://www.atcc.org/products/crl-2117)

The methods provided herein may be used with one type of cell to create a variety of products. In some embodiments, the cells cultivated in step (a) are embryonic stem cell-derived cells or primary cells. In some embodiments, the cells are embryonic stem-cell derived. In some embodiments, the embryonic stem-cell derived cells are derived from fish embryos. In some embodiments, the fish embryos are from any suitable edible fresh and salt-water fish and aquatic animals. In some embodiments, the cells are primary cells. In some embodiments, the primary cells are derived from larval or adult fish tissues. In some embodiments, the larval or adult fish tissues are from any suitable edible fresh and salt-water fish and aquatic animals.

According to some embodiments, there is provided a method for culturing a cell. In some embodiments, the cell is an animal cell. In some embodiments, the cell is derived from an aquatic organism. In some embodiments, the cell is a mammalian cell. According to some embodiments, there is provided a method for culturing a cell derived from an aquatic organism. In some embodiments, the aquatic organism is an aqua cultured organism. In some embodiments, the aquatic organism is a fished organism, e.g., obtained by fisheries. In some embodiments, the aquatic organism is selected form: a mollusk, a fish, a crustacean, a phytoplankton, or a zooplankton.

For instance, primary cells can be isolated from the following species: Pecten jacobaeus; Ostrea edulis; Spondylus spinosus; Pecten maximus; Argopecten irradians; Placopecten magellanicus; and Aequipecten opercularis.

Isolation and establishment of primary cells were performed according to the following protocol. Brush the valves, washed in sterile seawater (SSW, 0.2 μm filter sterilized) and rinsed with 70% ethanol. Open scallops carefully and rinse the inside twice with 20 ml of SSW. Remove the heart, adductor muscle and mantel with sterile forceps and dissected into 1-3 mm pieces. Wash tissue pieces 3 times with antibiotics in decreasing concentrations of 4×, 2×, and 1× (antibiotics 1× was a solution of penicillin-streptomycin μg/ml), kanamycin (10 μg/ml), and gentamycin (20 μg/ml) in SSW. Minced tissue pieces are suspended in 0.025-0.1% pronase, or trypsin in Hanks' balanced salt solution (HBSS, osmolality 1,060-1, 100 mOsm) for 12 hat 4° C. After dissociation, the cells are filtered through a 60 μm nylon mesh and centrifuged at 300 g for 5 min. Pellet is resuspended in SSW containing 10% fetal bovine serum (FBS) and washed twice with SSW to stop enzymatic digestion. Cells are then resuspended in an appropriate culture medium and seeded at a density of 2.5×10⁶ per well, in 24-well plates in a final volume of 0.5 ml medium per well and incubated at 15° C. Half of the medium is replaced for the first time 2 days after seeding.

For instance, for embryonic cell cultures, embryonic material is obtained from artificial egg fertilization of the animals according to the following protocol. Shell surfaces are disinfected with 70% ethanol, and the animals (males and females separately) are placed in 14-15° C. UV SSW. Spawning is induced by injecting 0.5-2.0 ml of 10-3 M serotonin creatin sulfate into the gonad or adductor muscle. Sperm suspension from a few males is added to eggs (2-3 spermatozoa per one egg to avoid polyspermy) under microscopic control. The fertilized eggs are washed to remove spermatozoa, and are cultivated in closed tanks with SSW, which are stirred and constantly aerated with sterile air. Twenty hours after fertilization, larvae of the swimming-blastula stage are transferred into fresh SSW. Developed trochophore larvae are collected on a fine 30 μm mesh nylon screen, rinsed in sterile artificial Ca2+ and Mg2+ free sea water and concentrated to a small volume (0.5-1 ml) by centrifugation (1,000×g for 10 min, 5° C.). Larvae are treated in either 0.125% collagenase, pronase, or trypsin, and are dissociated completely for a period of 1.0-1.5 h at 10-12° C. Resulting cell suspension is washed twice in SSW with the antibiotic mix followed by centrifugation (1,000 g for 5 min), and pellet is resuspended in appropriate growth medium. Cell suspension of 0.5-1.0×106 cells/ml is dispensed into either collagen, gelatin, laminin or non-treated coated 24 well plates. Medium is changed after 24 hours.

The cultivation of cells (embryonic stem cell-derived or primary cells) can begin by thawing a frozen ampule of cells. The thawed cells are cultured and expanded until there are enough cells to be seeded in a bioreactor, such as a stirred tank bioreactor. The expanded cells can then be further cultivated in full suspension in the stirred tank bioreactor. In some embodiments, the cultivation of cells is performed in a continuous culture, fed-batch culture, repeated fed-batch culture, or batch culture. In some embodiments, the cultivation of cells is performed in a continuous culture.

In some embodiments, the cultivation of cells is performed in a continuous culture. Continuous culture is a method of cultivation in which an equilibrium is established. Under continuous culture, fresh culture medium is added (replenishing nutrients and carbons sources) as it is removed (removing cellular waste and medium depleted of nutrients). When the depleted culture medium is removed, cells can also be harvested. In some embodiments, the cells can be retained in a perfusion process. Perfusion can involve using methods (such as a filter membrane) to keep cells in a bioreactor while continuously exchanging culture medium or to recycle cells back to the bioreactor. The advantages of using continuous culture include maximizing productivity as well as reducing the time and effort for cleaning, sterilization and handling of the bioreactor vessel between cultures.

In some embodiments, a continuous culture process lasts, for each run, between 2-12 months, 2-10 months, 2-8 months, 2-6 months, 2-4 months, 4-12 months, 4-10 months, 4-8 months, 4-6 months, 6-12 months, 6-10 months, 6-8 months, 8-12 months, 8-10 months or 10-12 months, each inclusive. In some embodiments, a continuous culture process lasts, for each run, between 2-8 months.

In some embodiments, the cultivation of cells is performed in a fed-batch culture. In a fed-batch culture, fresh media can be introduced during the partly open cultivation process to achieve increased resulting biomass and product yield. The user can manually set the introduction of fresh media at a specific time or add the fresh media when specific conditions are met (for example, when a certain resulting biomass is reached or when a certain nutrient is depleted). The advantage of using batch culture is that it can be manipulated to increase a culture's productivity (e.g., achieve higher product quantity) and used with different feeding strategies. The disadvantage include an accumulation of by-products and waste and introduce the possibility for contamination.

In some embodiments, the cultivation of cells is performed in a repeated fed-batch culture. In some embodiments, a repeated fed-batch culture is also referred to as a semi-continuous culture. A repeated fed-batch culture can involve harvesting all the cells but leaving a small portion resulting cells from the previous culture for the next fed-batch culture. The remaining culture is supplied with fresh media, and the process can be repeated over several cycles. Compared to a fed-batch, a repeated fed-batch prevents the accumulation of by-products and waste.

In some embodiments, the cultivation of cells is performed in a batch culture. In a batch culture, no extra supply of fresh culture medium is further introduced once the initial media is supplied. All nutrients are provided at the beginning of the cultivation process. No additional nutrients are added to the closed system once the cultivation begins.

### II. Harvesting Methods

In some embodiments, the cultivation of cells is performed in a continuous culture. Cells can be harvested as raw materials for food products. In some embodiments, the cells can be harvested at between about 1-7 days, 1-6 days, 1-5 days, 1-4 days, 1-3 days 1-2 days, 2-7 days, 2-6 days, 2-5 days, 2-4 days, 2-3 days, 3-7 days, 3-6 days, 3-5 days, 3-4 days, 4-7 days, 4-6 days, 4-5 days, 5-7 days, 5-6 days or 6-7 days, each inclusive.

In some embodiments, the cultivated cells are harvested at least once a day. In some embodiments, the cells can be harvested at between about 2-24 hours, 2-20 hours, 2-16 hours, 2-12 hours, 2-8 hours, 2-4 hours, 4-24 hours, 4-20 hours, 4-16 hours, 4-12 hours, 4-8 hours, 8-24 hours, 8-20 hours, 8-16 hours, 8-12 hours, 12-24 hours, 12-20 hours, 12-16 hours, 16-24 hours, 16-20 hours or 20-24 hours. In some embodiments, the cells can be harvested at between about every 8-24 hours. In certain embodiments, the harvesting of cultivated cells occurs between about every 8-20 hours. In certain embodiments, the harvesting of cultivated cells occurs between about every 8-16 hours. In certain embodiments, the harvesting of cultivated cells occurs between about every 8-12 hours. In some embodiments, the harvesting of cultivated cells occurs at or about every 2 hours, 4 hours, 8 hours, 16 hours, 20 hours or 24 hours. In certain embodiments, the harvesting of cultivated cells occurs at or at about every 16 hours. In certain embodiments, the harvesting of cultivated cells occurs at or at about every 24 hours. In certain embodiments, the harvesting of cultivated cells occurs at or at about every 12 hours.

In some embodiments, the cultivated cells are harvested no more than 6 times a day. In some embodiments, the cultivated cells are harvested 1-6 times, 1-5 times, 1-4 times, 1-3 times, 1-2 times, 2-6 times, 2-5 times, 2-4 times, 2-3 times, 3-6 times, 3-5 times, 3-4 times, 4-6 times, 4-5 times, or 5-6 times a day, each inclusive. In some embodiments, the cultivated cells are harvested 1-3 times a day. In some embodiments, the cultivated cells are harvested no more than 6 times a day, 5 times a day, 4 times a day, 3 times a day, 2 times a day, or 1 time a day.

In some embodiments, the harvesting of cultivated cells harvests between about 20-80 percent, 20-70 percent, 20-60 percent, 20-50 percent, 20-40 percent, 20-30 percent, 30-80 percent, 30-70 percent, 30-60 percent, 30-50 percent, 30-50 percent, 30-40 percent, 40-80 percent, 40-70 percent, 40-60 percent, 40-50 percent, 50-80 percent, 50-70 percent, 50-60 percent, 60-80 percent, 60-70 percent and 70-80 percent of the content of a bioreactor, each inclusive. In some embodiments, the harvesting of the cultivated cells harvests between about 30 to 70 percent of the content of a bioreactor. In some embodiments, the harvesting of the cultivated cells harvests at or about 20%, 30%, 40%, 50%, 60%, 70% or 80% of the contents of a bioreactor. In some embodiments, the harvesting of cultivated cells harvests at or about 70 percent of the content of a bioreactor. In some embodiments, the harvesting of the cultivated cells harvests at or about 50% of the contents of a bioreactor. In some embodiments, the harvesting of cultivated cells harvests at or about 30 percent of the content of a bioreactor. In certain embodiments, the harvesting of cultivated cells harvests a steady flow of cells, such as in a method known in the art as “bleeding”.

In some embodiments, a bioreactor can be any vessel for culture and expansion of cells. A bioreactor can refer to a manufactured device of system that can support a biologically active environment for growing cells. In some embodiments, the bioreactor can comprise bioreactors, tanks, columns and/or tubes.

In some embodiments, the harvested, cultivated cells are separated from spent media using any suitable methods known in the art. In some variations, the harvested, cultivated cells are separated from spent media using centrifugation. In other variations, other suitable methods may include alternating tangential flow filtration (ATF) or tangential flow filtration (TFF). In yet other variations, the harvested, cultivated cells are separated from spent media using rolling drums separation, decanter centrifuge, or rotary vacuum-drum filter.

The spent media can be recycled and supplemented with algae-extracted components using any of the methods described herein.

In some embodiments, the harvested, cultivated cells are unwashed or washed after the separation from the spent media. In some embodiments, if the residual media is safe for consumption by a human, the harvested, cultivated cells are unwashed after the separation and used as they are. A skilled artisan in the field of food safety would be able to determine if the residual is safe for human consumption. The harvested, cultivated cells, with the residual media, can be used a raw material for food production.

In some embodiments, the harvested, cultivated cells are washed after the separation from the spent media. The washing may be necessary to prepare the harvested, cultivated cells' use as a raw material for food production. In some embodiments, the washing is performed in an isotonic solution and/or water. In some embodiments, the washing is performed in an isotonic solution and water. In some embodiments, the washing is performed in an isotonic solution. In some embodiments, the washing is performed in water. An isotonic solution can be a solution that has the same solute concentration and water concentration compared to human bodily fluids. Examples of isotonic solutions can include, but are not limited to, normal saline, lactated Ringer's solution, 5% dextrose in water or Ringer's solution.

In some embodiments, the cells to be used as raw material for food production can be washed no more than 6 times, 5 times, 4 times, 3 times, 2 times or 1 time. In some embodiments, the cells to be used as raw material for food production can be washed at least 1 time, at least 2 times, at least 3 times, at least 4 times, at least 5 times, or at least 6 times. In some embodiments, the cells to be used as a raw material for food production can be washed between 1-6 times, 1-5 times, 1-4 times, 1-3 times, 1-2 time, 2-6 times, 2-5 times, 2-4 times, 2-3 times, 3-6 times, 3-5 times, 3-4 times, 4-6 times, 4-5 times or 5-6 times.

In some embodiments, the harvested cultivated cells are used as fresh raw materials for cultivated food production.

In some embodiments, the harvested cells are frozen for later use. In some embodiments, the harvested cells are washed are described herein before being frozen for later use. In some embodiments, the harvested cells are unwashed before being frozen for later use.

In some embodiments, the harvested cells are frozen with or without a cryoprotectant. In some embodiments, the harvested cells are frozen with a cryoprotectant. In some embodiments, the harvested cells are frozen without a cryoprotectant.

In some embodiments, the cryoprotectant is a food grade cryoprotectant. In some variations, the cryoprotectant comprises suitable polyhydroxy compounds. In certain variations, the cryoprotectant comprises suitable sugars, sugar alcohols, and phosphates. In some embodiments, the food grade cryoprotectant can include, but is not limited to, acacia gum, acetic acid, adenine, alkoxylated fatty acid ester (vegetable), ammonium chloride, ammonium citrate (dibasic), ammonium hydroxide, ascorbic acid, betaine, calcium ascorbate, calcium carbonate, calcium chloride, calcium phosphate dibasic, casein enzymatic hydrolysate, casein hydrolysate, casein peptone, citric acid, corn starch, corn syrup solids, cysteine L, cysteine monohydrochloride L, dextrose monohydrate, diammonium phosphate, dimethylpolysiloxane, dipotassium hydrogen phosphate, disodium inosinate, formic acid, fructose, glutamic acid L, glycerol, glycine, inosine, inositol (vitamin B8), lactose, lysine, magnesium sulfate, maltitol, maltodextrin, maltose (hydrogenated), manganese chloride, manganese sulphate monohydrate, mannitol, microcrystalline cellulose, milk powder, monoammonium phosphate, monopotassium phosphate, monosodium glutamate L, nitrogen (liquid), phosphoric acid, polysorbate 80, potassium citrate, potassium hydroxide, potato starch, propyle gallate, rice flour, rice protein hydrolysate, silicon dioxide, skim milk powder, sodium alginate, sodium ascorbate/ascorbic acid, sodium aspartate/aspartic acid, sodium bicarbonate, sodium caseinate, sodium chloride, sodium citrate (di- and tri-), sodium dodecyl sulfate, sodium formate, sodium hydroxide, sodium lauryl sulfate, sodium phosphate (monobasic), sorbitol, soy lecithin, soy peptone, starch, sucrose/saccharose, trehalose, trisodium citrate dihydrate, whey (powder), whey protein, yeast peptone, yeast/yeast extract, and zein from corn. In some embodiments, the food grade cryoprotectant is selected from the group consisting of: fructose, glycerol, lactose, skim milk, sodium phosphate, sorbitol, sucrose/saccharose, and trehalose. In some embodiments, the food grade cryoprotectant is fructose. In some embodiments, the food grade cryoprotectant is glycerol. In some embodiments, the food grade cryoprotectant is lactose. In some embodiments, the food grade cryoprotectant is sodium phosphate. In some embodiments, the food grade cryoprotectant is sorbitol. In some embodiments, the food grade cryoprotectant is sucrose/saccharose. In some embodiments, the food grade cryoprotectant is trehalose. In other variations, the cryoprotectant does not include any animal-derived components.

### III. Recycling Spent Media

The cultured meat and seafood industry is a relatively new industry aiming at the generation of cultured meat and seafood products obtained by the cultivation of cells originated from different organisms using commonly accepted cell culture techniques. There are numerous companies in the field each of which differing from the other mainly by the organism of choice. However, the basic underlying principal is pretty much shared among all companies, that is cells are obtained from a biopsy, cells are then grown in a bioreactor to high densities, and finally the cells are harvested to create a hybrid product (plant scaffold infused with cells). During this critical growth phase, the cells are producing waste products (e.g., ammonia, lactate, CO₂, phosphates, and others) which are mixed with the growth media rendering it toxic, even though there are still vital nutrients in the spent medium. This leads to extensive media replacement and to excessive amounts of growth media needed for the proliferation of the cells, making the process extremely expensive, wasteful, and detrimental to the environment. This basic issue is shared among all of the cell-based industries. Therefore, there is a growing need for the development of novel technologies for the treatment and recycling of cells spent media for the cultured meat and seafood industry as well as for other cellular based industries.

Animal cells exploit nutrients from culture media, and while doing so, they excrete ammonia and other secondary metabolites that are harmful to cells, slow down cell division, and may even cause cell death in high concentrations. See Silvac et al. Cytotechnology (2010) 52:585-594. Typically, the waste medium is discarded and replaced with a new culture medium, leading to large volumes of discarded waste medium which can lead to environmental problems. Additionally, the volumes of fresh media necessary to replenish the culture media also adds additional cost. As a result, there is a need for methods to figure out recycling of spent culture media to remove by-products of cell cultivation, such as ammonia and phosphate, and re-use the spent media for continued cell cultivation.

Microalgae are a diverse and versatile group of microorganisms that inhabit different types of environments from sea water to fresh water and can withstand a wide range of environmental conditions. These unique organisms convert CO₂ and waste products (primarily ammonia) into algal biomass bloom which could be used in numerous ways (fertilizer, food additive, cosmetics, biofuel industry and more).

The use of algae systems is extensive, and these systems have been used for a long time by different industries to treat different types of wastewaters produced by industrial systems primarily aquaculture, sewage systems and more. Given all the above, microalgae are a prime candidate to serve as a key component of waste management systems, one such example is the use of microalgae to filter waste products produced in the aquafarming industry, also known as aquaponics.

According to some embodiments, there is provided a method for treating spent cell culture medium. In some embodiments, the method of the invention is directed to providing or obtaining a treated spent cell culture medium.

In some embodiments, the method comprises contacting the spent cell culture medium with an effective amount of microalgae.

In some embodiments, the spent cell cultured medium was used for culturing a cell. In some embodiments, the cell is an animal cell. In some embodiments, the cell is obtained or derived from an animal. In some embodiments, the cell is a primary cell culture. In some embodiments, the cell is an immortalized cell. In some embodiments, the cell is a cell line. In some embodiments, the cell is a stem cell, such as, but not limited to an embryonic stem cell.

In some embodiments, the animal cell is derived or obtained from any one of an invertebrate organism or a vertebrate organism.

In some embodiments, the invertebrate organism is selected from: mollusc, crustacean, and insect.

In some embodiments, the invertebrate organism is an arthropod.

In some embodiments, the insect is a moth or a fly.

In some embodiments, the vertebrate organism is selected from: mammal, fish, or avian.

In some embodiments, the mammal is selected from: primate, bovine, rodent, and porcine.

In some embodiments, the avian is a chicken or a duck.

In some embodiments, the primate is a human.

In some embodiments, treating comprises reducing the concentration of at least one compound selected from: ammonia, CO₂, phosphate, lactate, or any combination thereof, in the spent cell culture medium. In some embodiments, reducing is by at least 5%, at least 15%, at least 25%, at least 35%, at least 50%, at least 75%, at least 85%, at least 90%, at least 95%, at least 99%, or 100% reducing, or any value and range therebetween. Each possibility represents a separate embodiment of the invention.

In some embodiments, the method further comprises a step comprising culturing a cell on the treated spent culture medium.

In some aspects, provided herein are methods of recycling spent media from the cultivation of cells for cultivated food production. In some embodiments, the method comprises: (a) separating the spent media from the cultured cells; (b) introducing microalgae to the spent media; (c) cultivating the microalgae to recycle the spent media; and (d) separating the microalgae from the recycled media. The spent media from the culturing of cells usually contains high amounts of ammonia and phosphate. In some embodiments, the method comprises cultivating microalgae in the spent media because the microalgae can utilize the ammonia and phosphate (e.g., recycle), enabling the recycling of the spent cell culture media. In some embodiments, the methods described herein further comprises adding microalgae-derived components to adjust the recycled media formulation. The recycling methods provided herein can be used for the cultivation of any type of cells for the development of food products.

In some embodiments, the cultured cells in step (a) are embryonic stem cell-derived cells or primary cells. In some embodiments, the cells are embryonic stem-cell derived. In some embodiments, the embryonic stem-cell derived cells are derived from fish embryos. In some embodiments, the fish embryos are from any suitable edible fresh and salt-water fish and aquatic animals. In some embodiments, the cells are primary cells. In some embodiments, the primary cells are derived from larval or adult fish tissues. In some embodiments, the larval or adult fish tissues are from any suitable edible fresh and salt-water fish and aquatic animals.

In some embodiments, the genus of microalgae in step (b) can include, for example, Chlorella, Phaeodactylum, Nannochloropsis, Isochrysis, Chlorococcum, Porphyridiophyceae, Dunaliella, Nannochloropsis, Tetraselmis and Arthrospira genera, and any combinations thereof. In particular embodiments, the genus of microalgae in step (b) can be selected from the group consisting of: Chlorella, Porphyridiophyceae, Dunaliella, Nannochloropsis and Tetraselmis. In some embodiments, the species of microalgae in step (b) can include, but are not limited to: Phaeodactylum tricornutum, Nannochloropsis oceanica, Isochrysis galbana, Spirulina, Chlorella ulgaris, Chlorella sp., Chlorococcum littorale, Arthrospira platensis, Porphyridium purporuim, Duneliela salina, Tetraselmis striata and any combinations thereof. In particular embodiments, the species of microalgae in step (b) can be selected from the group consisting of: Chlorella ulgaris, Chlorella sp., Porphyridium purporuim, Duneliela salina and Tetraselmis striata. In some embodiments, the microalgae are Phaeodactylum tricornutum. In some embodiments, the microalgae are Nannochloropsis oceanica. In some embodiments, the microalgae are Chlorella vulgaris. In other embodiments, the genus of microalgae in step (b) can include Chlorococcum littorale; Chlorococcum submarinum; Porphyridium purpureum; Dunaliella salina; Dunaliella sp.; Chlorella vulgaris; Chlorella sp.; Arthrospira platensis; Euglena gracilis; Acutodesmus obliquus; Lobosphaera incisa; Tetraselmis striata; Tetraselmis sp.; Diacronema lutheri; Nannochloropsis oculate; Nannochloropsis sp.; Porphyridium purpureum (P. p); Isochrysis galbana (I. g); Phaeodactylum tricornutum (P. t); and Haematococcus pluvialis.

Microalgae are typically considered photoautotrophic organisms-they are photosynthetic organisms that utilize energy light energy and inorganic carbons to synthesize organize compounds and nutrients. In some embodiments, the microalgae is cultivated in an autotrophic, phototrophic, heterotrophic, or mixotrophic cultivation conditions. In some embodiments, the microalgae are cultivated in an autotrophic condition. Autotrophic organisms produce their own food from the substances in their surroundings using light, water, carbon dioxide or other chemicals. Microalgae, when grown in autotrophic conditions, use energy from light and carbon dioxide to grow. In some embodiments, the microalgae are cultivated under a phototrophic condition. Phototrophic organisms can use energy from light to carry out various cellular and/or metabolic processes. Microalgae, when grown in phototrophic conditions, use energy from light and carbon dioxide as a carbon source. In some embodiments, the microalgae are in a heterotrophic cultivation condition. Heterotrophic organisms cannot synthesize their own food and rely on other organisms, including plants and animals, for their nutrition. Microalgae, when grown in heterotrophic conditions, typically grown in the dark and use organic compounds (such as glucose or even waste sugars) as their carbon and energy sources. In some particular embodiments, heterotrophic conditions can contain about 2.2 g/L glucose; or between about 1-20 g/L glucose, 1-1.8 g/L glucose, 1-1.4 g/L glucose, 1.4-2.2 g/L glucose, 1.4-1.8 g/L glucose, 1.8-2.2 g/L glucose. In particular embodiments, heterotrophic conditions can contain between about 1.4-2.2 g/L glucose, each inclusive. In particular embodiments, heterotrophic conditions can contain about 1.8 g/L glucose. In some embodiments, the microalgae are cultured in an mixotrophic cultivation condition. Mixotrophic organisms can use a mix of different sources of energy and carbon for their growth and survival. Microalgae, when grown in mixotrophic conditions, typically are supplied both light energy and organic carbons. In particular embodiments, mixotrophic conditions can contain between about 1-20 g/L glucose, 1-15 g/L glucose, 1-10 g/L glucose, 1-5 g/L glucose, 5-20 g/L glucose, 5-15 g/L glucose, 5-10 g/L glucose, 10-20 g/L glucose, 10-15 g/L glucose or 15-20 g/L glucose, each inclusive.

In some embodiments the light energy is calculated by lux light intensity. In particular embodiments, the lux light intensity is between about 2,500-10,000 lux light intensity, 2,500-5,000 lux light intensity, 2,500-4,000 lux light intensity, 2,500-3,000 lux light intensity, 3,500-5,000 lux light intensity, 3,500-4,000 lux light intensity, or 4,500-5,000 lux light intensity, each inclusive. In specific embodiments, the lux light intensity is or is about 3,000, 3,200, 3,400, 3,600 or 3,800 lux light intensity. In specific embodiments, the lux light intensity is or is about 3,200 lux light intensity.

For instance, to the test and demonstrate the ability of microalgae in purifying cell based spent media, selected microalgae are used to purify contaminated growth media, among which are: Phaeodactylum tricornutum—Seawater microalgae; Nannochloropsis oceanica—Seawater microalgae; Isochrysis galbana—Seawater microalgae; Spirulina—Freshwater and seawater microalgae; and Chlorella vulgaris—Freshwater microalgae.

Different types of spent media are adjusted to osmolarity fitting that of the tested microalgae with supplementation of critical elements, as well. Other than that, the spent media is used with minimal adjustments to simplify the process. For the media clearing capability, the inventors further disclose hereinafter two non-limiting parallel approaches: (i) Artificial spent media analysis-Reconstructing commonly used complete growth media with the addition of ammonia (5 mM) and lactate (3 mM; key waste products during cellular metabolism) at varying concentrations; and (ii) Cell originated spent media analysis-Collecting spent media from commonly used cell lines as described herein.

In some embodiments, the microalgae used in the recycling methods can include Chlorococcum littorale; Chlorococcum submarinum; Porphyridium purpureum; Dunaliella salina; Dunaliella sp.; Chlorella vulgaris; Chlorella sp.; Arthrospira platensis; Euglena gracilis; Acutodesmus obliquus; Lobosphaera incisa; Tetraselmis striata; Tetraselmis sp.; Diacronema lutheri; Nannochloropsis oculate; Nannochloropsis sp.; Porphyridium purpureum (P. p); Isochrysis galbana (I. g); Phaeodactylum tricornutum (P. t); and Haematococcus pluvialis.

In other embodiments, the microalgae used in the recycling methods provided herein is of the Chlorella genus. In some embodiments, the microalgae used in the recycling methods provided herein is Chlorella vulgaris. Under phototrophic conditions, chlorella vulgarius has been reported to have a doubling time of around 4.2 days. See El-Ibiari et al., International Journal of ChemTech Research (2015), 8 (9): 284-289. Under heterotrophic conditions, chlorella vulgarius tripled its cell density (starting from a density of 6×10⁷ cells/ml) when grown on spent media within two days. In some embodiments, the microalgae used in the recycling methods provided herein is Chlorella sp. The density of Chlorella sp. was 4.5 times higher, compared to its starting density at day 0 (starting from a density of 2×10⁷ cells/ml), after cultivation on spent media. See Levin et al., Plant Journal (2021), 106:1260-1277.

In some embodiments, the doubling time of the density of the microalgae is between about 1-5 days, 1-4 days, 1-3 days, 1-2 days, 2-5 days, 2-4 days, 2-3 days, 3-5 days, 3-4 days or 4-5 days, each inclusive. In some embodiments, the doubling time of the density of microalgae is between about 1-2 days. In some embodiments, the tripling time of the density of the microalgae is between about 1-5 days, 1-4 days, 1-3 days, 1-2 days, 2-5 days, 2-4 days, 2-3 days, 3-5 days, 3-4 days or 4-5 days, each inclusive. In some embodiments, the tripling time of the density of microalgae is between about 1-2 days.

In some embodiments, cell division of the microalgae is between about every 2-8 hours, 2-6 hours, 2-4 hours, 4-8 hours, 4-6 hours, or 6-8 hours, each inclusive. In some embodiments, cell division of the microalgae is between about every 2 to 6 hours, inclusive. In some embodiments, the cell division of the microalgae is or is about every 4 hours. In some embodiments, the cell division of the microalgae is or is about every 5 hours.

In some embodiments, prior to the introduction of the microalgae to the spent media, the spent media is filtered to avoid cell debris.

In some embodiments, the spent media is transferred to a vessel containing microalgae. In some embodiments, the vessel containing microalgae is a bioreactor. In some embodiments, the vessel containing microalgae is a tank. In some embodiments, the vessel containing microalgae is comprised of columns and tubes.

In some embodiments, the vessel containing microalgae is set up under autotrophic, heterotrophic, phototropic, or mixotrophic conditions. In some variations, the vessel containing microalgae is set up under autotrophic, heterotrophic, or mixotrophic conditions.

In certain variations, the vessel containing microalgae is set up under autotrophic conditions. Under autotrophic conditions, the vessel containing microalgae is equipped with a light to serve as an energy source. In some embodiments, the vessel containing microalgae is set up under heterotrophic conditions. Under heterotrophic conditions, the vessel containing microalgae is in the dark. Growing in heterotrophic conditions offers an advantage due to the glucose remaining in the spent medium that the algae can grow on. Supplementing glucose can be easier than operating light and CO₂ aeration. In some embodiments, the vessel containing microalgae is set up under mixotrophic conditions. Under mixotrophic conditions, light is used as an energy source and CO₂ and organic carbon are used as the carbon sources.

Heterotrophic and mixotrophic growth conditions can be advantageous for a faster ammonia level reduction. The use of heterotrophic conditions can also be advantageous because there will be no undesirable exposure of light-sensitive molecules in the spent media to a light source. In some embodiments, the vessel containing microalgae is set up to preserve light sensitive molecules in the spent media. Light sensitive molecule preservation is advantageous because during the recycling process, the spent media is not compromised. When light-sensitive molecules are preserved, light sensitive molecules do not need to be added back into the recycled media. In some embodiments, the light sensitive molecules are hormones, vitamins, or growth factors. In some embodiments, the light sensitive molecules are hormones. In some embodiments, the light sensitive molecules are vitamins. In some embodiments, the light sensitive molecules are growth factors.

In some embodiments, the vessel containing microalgae comprises an effective amount of microalgae to perform the recycling of the spent media. The effective amount of microalgae can be grown in flasks at room temperature. In some embodiments, the growing of microalgae includes shaking between about 100-200 RPM, 100-180 RPM, 100-160 RPM, 100-140 RPM, 100-120 RPM, 120-200 RPM, 120-180 RPM, 120-160 RPM, 120-140 RPM, 140-180 RPM, 140-160 RPM, 160-200 RPM, 160-180 RPM or 180-200 RPM, each inclusive. In some embodiments, the growing of microalgae includes shaking between about 140-180 RPM. In some embodiments, the growing of microalgae includes shaking at or at about 100 RPM, 120 RPM, 140 RPM, 160 RPM, 180 RPM or 200 RPM. In some embodiments, the growing of microalgae includes shaking at or at about 160 RPM.

In some embodiments, the effective amount of microalgae comprises between about 1×10⁴ to 1×10¹⁰, between about 1×10⁴ to 1×10¹⁰, between about 1×10⁴ to 1×10⁸, between about 1×10⁴ to 1×10⁶, between about 1×10⁵ to 1×10⁹, between about 1×10⁵ to 1×10⁷, between about 1×10⁶ to 1×10¹⁰, between about 1×10⁶ to 1×10⁸, between about 1×10⁷ to 1×10⁹ or between about 1×10⁸ to 1×10¹⁰ microalgae cells per milliliter of spent media, each inclusive. In some embodiments, the effective amount of microalgae comprises between about 1×10⁷ to 1×10⁹ microalgae cells per milliliter of spent media, inclusive. In some embodiments, the effective amount of microalgae comprises about 5×10⁷ microalgae cells per milliliter of spent media. In some embodiments, the effective amount of microalgae comprises about 1×10⁸ microalgae cells per milliliter of spent media. In some embodiments, the effective amount of microalgae comprises about 1×10⁹ microalgae cells per milliliter of spent media.

In some embodiments, the effective amount of microalgae for recycling spent media is 1×10³ to 1×10⁹ microalgae cells per milliliter of spent media, inclusive. In particular embodiments, the effective amount of microalgae for recycling spent media is 1×10³ microalgae cells per milliliter of spent media. In particular embodiments, the effective amount of microalgae for recycling spent media is 1×10⁴ microalgae cells per milliliter of spent media. In particular embodiments, the effective amount of microalgae for recycling spent media is 1×10⁵ microalgae cells per milliliter of spent media. In particular embodiments, the effective amount of microalgae for recycling spent media is 1×10⁶ microalgae cells per milliliter of spent media. In particular embodiments, the effective amount of microalgae for recycling spent media is 1×10⁷ microalgae cells per milliliter of spent media. In particular embodiments, the effective amount of microalgae for recycling spent media is 1×10⁸ microalgae cells per milliliter of spent media. In particular embodiments, the effective amount of microalgae for recycling spent media is 1×10⁹ microalgae cells per milliliter of spent media.

In some embodiments, the microalgae reduce the concentration of at least one compound in the spent media selected from the group consisting of: ammonia, phosphate, lactate, glucose, and carbon dioxide. In some embodiments, the microalgae reduce the concentration of ammonia in the spent media. In some embodiments, the microalgae reduce the concentration of phosphate in the spent media. In some embodiments, the microalgae reduce the concentration of lactate in the spent media. In some embodiments, the microalgae reduce the concentration of glucose in the spent media. In some embodiments, the microalgae reduce the concentration of carbon dioxide in the spent media.

In some embodiments, an effective amount of microalgae is used to reduce the concentration of ammonia in the spent media. In some embodiments, the effective amount of microalgae comprises between about 1×10⁶ to 1×10⁸ microalgae cells per milliliter of spent media for two days of ammonia cleaning, inclusive. In some embodiments, the effective amount of microalgae comprises 1×10⁶ microalgae cells per milliliter of spent media for two days of ammonia cleaning. In some embodiments, the effective amount of microalgae comprises 1×10⁷ microalgae cells per milliliter of spent media for two days of ammonia cleaning. In some embodiments, the effective amount of microalgae comprises 1×10⁸ microalgae cells per milliliter of spent media for two days of ammonia cleaning.

In some embodiments, the treated spent culture medium is characterized by comprising ammonia in an amount or concentration of <100 mM, <50 mM, <30 mM, <20 mM, <10 mM, <8 mM, <7 mM, <6 mM, <5 mM, <4 mM, <3 mM, <2 mM, <1 mM, <0.1 mM, or any value and range there between. Each possibility represents a separate embodiment of the invention.

In some embodiments, the provided methods herein recycle between about 1 to 10 mM ammonia, between about 1 to 7 mM ammonia, between about 1 to 4 mM ammonia, between about 4 to 10 mM ammonia, between about 4 to 7 mM ammonia or between about 7 to 10 mM ammonia in 48 hours, each inclusive. In some embodiments, the provided methods herein recycle between about 1 to 10 mM ammonia in 48 hours, inclusive.

In some embodiments, the provided methods herein reduce the level of ammonia by between about 20 to 80 percent, between about 20 to 60 percent, between about 20 to 40 percent, between about 40 to 80 percent, between about 40 to 60 percent, between about 60 to 80 percent from the initial level of ammonia, each inclusive. In some embodiments, the provided methods herein reduce the level of ammonia by between about 20 to 80 percent from the initial level of ammonia, inclusive. In some embodiments, the provided methods herein reduce the level of ammonia by between about 20 to 60 percent from the initial level of ammonia, inclusive. In some embodiments, the provided methods herein reduce the level of ammonia by between about 20 to 40 percent from the initial level of ammonia, inclusive. In some embodiments, the provided methods herein reuse and recycle ammonia from the spent media. In some embodiments, the provided methods herein avoid the need to discard large volumes of waste medium containing ammonia.

In some embodiments, the concentration of ammonia in the spent media is reduced by about 20 to 100 percent, by about 20 to 80 percent, by about 20 to 60 percent, by about 20 to 40 percent, by about 40 to 100 percent, by about 40 to 80 percent, by about 40 to 60 percent, by about 60 to 100 percent, by about 60 to 80 percent, or by about 80 to 100 percent, each inclusive. In some embodiments, the concentration of ammonia is reduced by about 20 to 100 percent, inclusive. In some embodiments, the concentration of ammonia is reduced by about 20 to 80 percent, inclusive. In some embodiments, the concentration of ammonia is reduced by about 20 to 60 percent, inclusive. In some embodiments, the concentration of ammonia is reduced by about 20 to 40 percent, inclusive. In some embodiments, the concentration of ammonia is reduced by about 20 percent. In some embodiments, the concentration of ammonia is reduced by about 40 percent. In some embodiments, the concentration of ammonia is reduced by about 60 percent. In some embodiments, the concentration of ammonia is reduced by about 100 percent.

In some embodiments, the spent culture medium is characterized by being devoid of at least one compound selected from: ammonia, CO2, phosphate, lactate, or any combination thereof. In some embodiments, the treated spent culture medium comprises ammonia in an amount of 4 mM at most, 3 mM at most, 2 mM at most, 1 mM at most, 0.1 mM at most, 0.01 mM at most, 1 μM at most, 0.1 μM at most, 10 nM at most, or 1 nM at most, or any value and range therebetween. Each possibility represents a separate embodiment of the invention.

The microalgae can consume all the ammonia from the spent media within hours to days. In some embodiments, the reduction of ammonia is completed within 1 to 168 hours, 1 to 144 hours, 1 to 120 hours, 1 to 96 hours, 1 to 72 hours, 1 to 48 hours, or 1 to 24 hours, each inclusive.

In some embodiments, an effective amount of microalgae is used to reduce the concentration of phosphate in the spent media. Phosphate is essential for cell growth. However, high phosphate levels in cell culture can lead to osmotic and cellular stress. Consequently, e the use of microalgae to reduce phosphate, but still not eliminate it completely from the medium can be beneficial for cell growth on the treated spent media.

In some embodiments, the effective amount of microalgae comprises between about 1×10⁶ to 1×10⁸ microalgae cells per milliliter of spent media for two days of phosphate cleaning, inclusive. In some embodiments, the effective amount of microalgae comprises 1×10⁶ microalgae cells per milliliter of spent media for two days of phosphate cleaning, inclusive. In some embodiments, the effective amount of microalgae comprises 1×10⁷ microalgae cells per milliliter of spent media for two days of phosphate cleaning. In some embodiments, the effective amount of microalgae comprises 1×10⁸ microalgae cells per milliliter of spent media for two days of phosphate cleaning.

In some embodiments, the concentration of phosphate is reduced by about 20 to 100 percent, by about 20 to 80 percent, by about 20 to 60 percent, by about 20 to 40 percent, by about 40 to 100 percent, by about 40 to 80 percent, by about 40 to 60 percent, by about 60 to 100 percent, by about 60 to 80 percent, or by about 80 to 100 percent, each inclusive. In some embodiments, the concentration of phosphate is reduced by about 20 to 100 percent. In some embodiments, the concentration of phosphate is reduced by about 20 to 80 percent. In some embodiments, the concentration of phosphate is reduced by about 20 to 60 percent. In some embodiments, the concentration of phosphate is reduced by about 20 to 40 percent. In some embodiments, the concentration of phosphate is reduced by about 40 to 100 percent, inclusive. In some embodiments, the concentration of phosphate is reduced by about 40 to 80 percent, inclusive. In some embodiments, the concentration of phosphate is reduced by about 40 to 60 percent, inclusive. In some embodiments, the concentration of phosphate is reduced by about 60 to 100 percent, inclusive. In some embodiments, the concentration of phosphate is reduced by about 60 to 80 percent, inclusive. In some embodiments, the concentration of phosphate is reduced by about 80 to 100 percent, inclusive.

In some embodiments, the reduction of phosphate is completed within 1 to 168 hours, 1 to 144 hours, 1 to 120 hours, 1 to 96 hours, 1 to 72 hours, 1 to 48 hours, or 1 to 24 hours. In some embodiments, the reduction of phosphate is completed within 1 to 168 hours, each inclusive.

In some embodiments, the treated spent culture medium is characterized by comprising lactate in an amount or concentration of <35 mM, <30 mM, <25 mM, <20 mM, <15 mM, <10 mM, <5 mM, <3 mM, <2 mM, <1 mM, <0.1 mM, or any value and range therebetween. Each possibility represents a separate embodiment of the invention.

In some embodiments, an effective amount of microalgae is used to reduce the concentration of lactate in the spent media. In some embodiments, the concentration of lactate is reduced by about 20 to 100 percent, by about 20 to 80 percent, by about 20 to 60 percent, by about 20 to 40 percent, by about 40 to 100 percent, by about 40 to 80 percent, by about 40 to 60 percent, by about 60 to 100 percent, by about 60 to 80 percent, or by about 80 to 100 percent, each inclusive. In some embodiments, the concentration of lactate is reduced by about 20 to 100 percent, inclusive. In some embodiments, the concentration of lactate is reduced by about 20 to 80 percent, inclusive. In some embodiments, the concentration of lactate is reduced by about 20 to 60 percent, inclusive. In some embodiments, the concentration of lactate is reduced by about 20 to 40 percent, inclusive. In some embodiments, the concentration of lactate is reduced by about 40 to 100 percent, inclusive. In some embodiments, the concentration of lactate is reduced by about 40 to 80 percent, inclusive. In some embodiments, the concentration of lactate is reduced by about 40 to 60 percent, inclusive. In some embodiments, the concentration of lactate is reduced by about 60 to 100 percent, inclusive. In some embodiments, the concentration of lactate is reduced by about 60 to 80 percent, inclusive. In some embodiments, the concentration of lactate is reduced by about 80 to 100 percent, inclusive.

In some embodiments, the spent culture medium is characterized by being devoid of at least one compound selected from: ammonia, CO2, phosphate, lactate, or any combination thereof. In some embodiments, the treated spent culture medium comprises lactate in an amount of 4 mM at most, 3 mM at most, 2 mM at most, 1 mM at most, 0.1 mM at most, 0.01 mM at most, 1 μM at most, 0.1 μM at most, 10 nM at most, or 1 nM at most, or any value and range therebetween. Each possibility represents a separate embodiment of the invention.

In some embodiments, the reduction of lactate is completed within 1 to 168 hours, 1 to 144 hours, 1 to 120 hours, 1 to 96 hours, 1 to 72 hours, 1 to 48 hours, or 1 to 24 hours.

In some embodiments, an effective amount of microalgae is used to reduce the concentration of glucose in the spent media. In some embodiments, the concentration of glucose is reduced by about 20 to 100 percent, by about 20 to 80 percent, by about 20 to 60 percent, by about 20 to 40 percent, by about 40 to 100 percent, by about 40 to 80 percent, by about 40 to 60 percent, by about 60 to 100 percent, by about 60 to 80 percent, or by about 80 to 100 percent, each inclusive. In some embodiments, the concentration of glucose is reduced by about 20 to 100 percent, inclusive. In some embodiments, the concentration of glucose is reduced by about 20 to 80 percent, inclusive. In some embodiments, the concentration of glucose is reduced by about 20 to 60 percent, inclusive. In some embodiments, the concentration of glucose is reduced by about 20 to 40 percent, inclusive. In some embodiments, the concentration of glucose is reduced by about 40 to 100 percent, inclusive. In some embodiments, the concentration of glucose is reduced by about 40 to 80 percent, inclusive. In some embodiments, the concentration of glucose is reduced by about 40 to 60 percent, inclusive. In some embodiments, the concentration of glucose is reduced by about 60 to 100 percent, inclusive. In some embodiments, the concentration of glucose is reduced by about 60 to 80 percent, inclusive. In some embodiments, the concentration of glucose is reduced by about 80 to 100 percent, inclusive.

In some embodiments, the reduction of glucose is completed within 1 to 168 hours, 1 to 144 hours, 1 to 120 hours, 1 to 96 hours, 1 to 72 hours, 1 to 48 hours, or 1 to 24 hours, each inclusive.

In some embodiments, an effective amount of microalgae is used to reduce the concentration of carbon dioxide in the spent media. In some embodiments, the concentration of CO₂ is reduced by about 20 to 100 percent, by about 20 to 80 percent, by about 20 to 60 percent, by about 20 to 40 percent, by about 40 to 100 percent, by about 40 to 80 percent, by about 40 to 60 percent, by about 60 to 100 percent, by about 60 to 80 percent, or by about 80 to 100 percent, each inclusive. In some embodiments, the concentration of CO₂ is reduced by about 20 to 100 percent, inclusive. In some embodiments, the concentration of CO₂ is reduced by about 20 to 80 percent, inclusive. In some embodiments, the concentration of CO₂ is reduced by about 20 to 60 percent, inclusive. In some embodiments, the concentration of CO₂ is reduced by about 20 to 40 percent, inclusive. In some embodiments, the concentration of CO₂ is reduced by about 40 to 100 percent, inclusive. In some embodiments, the concentration of CO₂ is reduced by about 40 to 80 percent, inclusive. In some embodiments, the concentration of CO₂ is reduced by about 40 to 60 percent, inclusive. In some embodiments, the concentration of CO₂ is reduced by about 60 to 100 percent, inclusive. In some embodiments, the concentration of CO₂ is reduced by about 60 to 80 percent, inclusive. In some embodiments, the concentration of CO₂ is reduced by about 80 to 100 percent, inclusive.

In some embodiments, the reduction of CO₂ is completed within 1 to 168 hours, 1 to 144 hours, 1 to 120 hours, 1 to 96 hours, 1 to 72 hours, 1 to 48 hours, or 1 to 24 hours, each inclusive. In some embodiments, the reduction of CO₂ is completed within 1 to 168 hours, percent.

In some embodiments, the microalgae can also secrete components into the recycled media. In particular embodiments, the microalgae, when recycling the spent media, may excrete ethanol into the media to be recycled. In particular embodiments, the microalgae, when recycling the spent media, may excrete vitamin B2 (riboflavin) into the media to be recycled.

In some embodiments, the recycling of the spent media comprises separating the microalgae from the recycled media. The microalgae and recycled media can be separated by centrifugation. In some embodiments, the centrifugation occurs at room temperature. In some embodiments, the centrifugation is between 1,000 to 7,000 g, 1,000 to 5,000 g, 1,000 to 3,000 g, 3,000 to 7000 g, 3,000 to 5,000 or 5,000 to 7,000 g, each inclusive. In particular embodiments, the centrifugation occurs at or at about 5,000 g. In particular embodiments, the centrifugation occurs at or at about 2,000 g. In particular embodiments, the centrifugation occurs at 2,300 g for 10 minutes. The separated microalgae can be in the form of a microalgae pellet.

In some embodiments, separation of the microalgae from the recycled media may be performed using any suitable methods known in the art. In certain embodiments, the separation of the microalgae from the recycled media is by centrifugation or by liquid-solid particle separation. In some embodiments, the separation of the microalgae from the recycled media is by centrifugation. In some embodiments, the separation of the microalgae from the recycled media is by liquid-solid particle separation. In some embodiments, the liquid-solid particle separation is performed by a rotary vacuum-drum filter. In other variations, other suitable methods may include alternating tangential flow filtration (ATF) or tangential flow filtration (TFF). In yet other variations, the harvested, cultivated cells are separated from spent media using rolling drums separation, decanter centrifuge, or rotary vacuum-drum filter.

In some embodiments, the separated microalgae from the recycled media is separated into at least two parts. In some embodiments, the separated microalgae from the recycled media is separated into two parts.

In some embodiments, the recycled media that is separated from the microalgae is further characterized for being depleted by about 20% to 100% of at least one compound selected from the group of: ammonia, phosphate, lactate, glucose, and carbon dioxide. In some embodiments, the recycled media that is separated from the microalgae is further characterized for being depleted by about 20% to 100% of ammonia. In some embodiments, the recycled media that is separated from the microalgae is further characterized for being depleted by about 40% to 100% of phosphate. In some embodiments, the recycled media that is separated from the microalgae is further characterized for being depleted by about 20% to 100% of lactate. In some embodiments, the recycled media that is separated from the microalgae is further characterized for being depleted by about 20% to 100% of glucose. In some embodiments, the recycled media that is separated from the microalgae is further characterized for being depleted by about 20% to 100% of carbon dioxide.

In some embodiments, the recycled media that is separated from the microalgae can be additionally depleted by about 20 to 100% of the following components, such as, but not limited to, arginine, vitamin B6 (nicotinamide), manganese and organic acid pyruvate. In particular embodiments, the recycled media that is separated from the microalgae can be additionally depleted by about 20 to 100% of the amino acid arginine. In particular embodiments, the recycled media that is separated from the microalgae can be additionally depleted by about 20 to 100% of the vitamin B6 (nicotinamide). In particular embodiments, the recycled media that is separated from the microalgae can be additionally depleted by about 20 to 100% of manganese. In particular embodiments, the recycled media that is separated from the microalgae can be additionally depleted by about 20 to 100% of the organic acid pyruvate.

### IV. Extracting Microalgae and Enriching Growth Media

Recent studies have shown that microalgae extracts can rescue to some degree pharma grade molecules commonly used in the industry like glucose and certain amino acids and maintain viability of mammalian cells in reduced serum conditions. This is particularly of importance to the cultured fish and seafood industry, where besides the need to reduce media costs, there is a need to emulate the distinct and unique flavors and nutritional profiles of these aquatic animals. These flavors and nutritional profiles originate from molecules produced by algae which are at the base of the aquatic food chain of mollusca, crustacea, fish, and other aquatic vertebrates and invertebrates. Algal lysates which are rich in bio-essential molecules such as vitamins, minerals, long chain fatty acids, and volatile compounds, are responsible for the highly nutritional value and certain flavor development related to aquatic animals.

There is still a great need for growth media formulated with algae lysates, which will create a superior cell-based raw material in terms of nutritional values and flavors. Algae species used for media algae extract derivation can include, but are not limited to: Chlorella vulgaris; Phaeodactylum tricornutum; Nannochloropsis oceanica; Isochrysis galbana; Spirulina; Chlorococcum littorale; and Arthrospira platensis.

Microalgae can produce nutrients, such as lipids, fatty acids, proteins, carbohydrates, fibers starches, antioxidants and nucleic acids. In some embodiments, the microalgae used in the microalgae nutrient extraction process is the microalgae used for recycling the spent media as described in Section III. In some embodiments, the microalgae used in the microalgae nutrient extraction process is a separate microalgae biomass, different from those described in Section III.

In some aspects, provided herein are methods of extracting microalgae derived components. In some embodiments, the method comprises: (a) lysing microalgae cells, wherein the lysing is performed by high-pressure homogenization; (b) treating the lysed microalgae cells with a detergent to release soluble proteins; (c) centrifuging the lysed microalgae cells treated with detergent; (d) collecting the supernatant; and (e) concentrating the extracted microalgae derived components from the supernatant by centrifugation. In some embodiments, the extracted microalgae derived components are filtered. In some embodiments, the microalgae derived components are added to a cell medium to enrich the cell medium. In other aspects, step (a) of lysing microalgae cell may further comprising sonication, chemical lysing, or heating. In yet other aspects, step (a) of lysing microalgae cell comprises alternative approaches to high-pressure homogenization, and employs methods and techniques such as sonication, chemical lysing, or heating.

In some embodiments, the microalgae-derived components of the enriched media in step (a) are derived from the same or different microalgae used for recycling the spent media in step (b). In some embodiments, the microalgae-derived components of the enriched media in step (a) are derived from the same microalgae used for recycling the spent media in step (b). In some embodiments, the microalgae-derived components of the enriched media in step (a) are derived from different microalgae used for recycling the spent media in step (b).

In some embodiments, the algae separated from the recycled media, as described in Section III, is divided into one part containing the original mass of algae for another cycle of spent media recycling and into another part that is lysed in preparation for the extraction of nutrients that will be added to preparation of enriched culture media. In some embodiments, the first part of the at least two parts of the separated microalgae from the recycled media is placed back into the vessel containing microalgae for at least one other cycle. In some embodiments, the first part of the at least two parts of the separated microalgae from the recycled media is placed back into the vessel containing microalgae for between about 1-9 other cycles, between about 1-6 other cycles, between about 1-3 other cycles, between about 3-9 other cycles, between about 3-6 other cycles, or between about 6-9 other cycles, each inclusive. In some embodiments, the first part of the at least two parts of the separated microalgae from the recycled media is placed back into the vessel containing microalgae for between about 1-9 other cycles. In some embodiments, the first part of the at least two parts of the separated microalgae from the recycled media is placed back into the vessel containing microalgae for 9 other cycles. In some embodiments, the first part of the at least two parts of the separated microalgae from the recycled media is placed back into the vessel containing microalgae for 6 other cycles. In some embodiments, the first part of the at least two parts of the separated microalgae from the recycled media is placed back into the vessel containing microalgae for 3 other cycles. In some embodiments, the first part of the at least two parts of the separated microalgae from the recycled media is placed back into the vessel containing microalgae for 1 other cycle.

In some embodiments, the second part of the at least two parts of the separated microalgae from the recycled media is frozen. In some embodiments, the second part of the at least two parts of the separated microalgae from the recycled media is stored between about −20° C. to −80° C., between about −20° C. to −60° C., between about −20° C. to −40° C., between about −40° C. to −80° C., between about −40° C. to −60° C., or between about −60° C. to −80° C., each inclusive. In some embodiments, the second part of the at least two parts of the separated microalgae from the recycled media is stored between about −20° C. to −80° C., inclusive. In some embodiments, the second part of the at least two parts of the separated microalgae from the recycled media is stored between about −20° C. to −60° C., inclusive. In some embodiments, the second part of the at least two parts of the separated microalgae from the recycled media is stored between about −20° C. to −40° C., inclusive. In some embodiments, the second part of the at least two parts of the separated microalgae from the recycled media is stored at or at about −80° C. In some embodiments, the second part of the at least two parts of the separated microalgae from the recycled media is stored at or at about −60° C. In some embodiments, the second part of the at least two parts of the separated microalgae from the recycled media is stored at or at about −40° C. In some embodiments, the second part of the at least two parts of the separated microalgae from the recycled media is stored at or at about −20° C.

In some embodiments, the second part of the at least two parts of the separated microalgae from the recycled media is extracted for the microalgae derived components.

In some variations, the process of microalgae nutrient extraction from the microalgae can involve the lysing or bursting of the algae cells in isotonic solution or water using high-pressure homogenizer, the addition of a detergent to the extract for better release of soluble proteins, centrifugation, and supernatant collection.

After the recycling described in Section III, the microalgae, if in pelleted form, can be re-dispersed in distilled water and or phosphate buffered saline solution. In some embodiments, the microalgae may be in slurry form and an additional step to re-disperse the cells may not be necessary. In some embodiments, the extraction of the microalgae derived components comprises lysing the microalgae cells. In some embodiments, the lysing of the microalgae cells comprises homogenizing the microalgae cells in isotonic solution and/or water. In some embodiments, the lysing of the microalgae cells comprises homogenizing the microalgae cells in isotonic solution. In some embodiments, the lysing of the microalgae cells comprises homogenizing the microalgae cells in water.

In some embodiments, the homogenization of the microalgae cells comprises the use of high-pressure homogenization. In some embodiments, the homogenization pressure is between about 1 to 80 kpsi, between about 1 to 60 kpsi, between about 1 to 40 kpsi, between about 1 to 20 kpsi, between about 20 to 80 kpsi, between about 20 to 60 kpsi, between about 20 to 40 kpsi, between about 40 to 80 kpsi, between about 40 to 60 kpsi, or between about 60 to 80 kpsi, each inclusive. In some embodiments, the homogenization pressure is between about 10 to 70 kpsi, inclusive. In some embodiments, the homogenization pressure is between about 20 to 60 kpsi, inclusive. In some embodiments, the homogenization pressure is between about 30 to 40 kpsi, inclusive.

In some embodiments, the homogenization pressure required to burst the microalgae cells is between about 1 to 50 psi, between about 1 to 45 psi, between about 1 to 40 psi, between about 5 to 50 psi, between about 5 to 50 psi, or between about 5 to 45 psi. In particular embodiments, the homogenization pressured required to burst the microalgae cells is between about 5 to 45 psi.

After the high-pressure homogenization, the lysed microalgae cells may be centrifuged. In some embodiments, the homogenized microalgae cells are centrifuged at between about 1000 g to 20,000 g, between about 1000 to 15,000 g, between about 1 to 10,000 g, between about 1 to 5,000 g, between about 5,000 to 20,000 g, between about 5,000 to 15,000 g, between about 5,000 to 10,000 g, between about 10,000 to 20,000 g, between about 10,000 to 15,000 g, or between about 15,000 to 20,000 g, each inclusive.

In some embodiments, the homogenized microalgae cells are centrifuged at between about 1° C. to 5° C., between about 1° C. to 10° C. between about 1° C. to 15° C., between about 5° C. to 10° C., between about 5° C. to 15° C. or between about 10° C. to 15° C., each inclusive.

In some embodiments, the centrifugation of the algae extract, after the bursting of the microalgae cells with the high-pressure homogenization, is between about 1,000 g to 200,000 g, is between about 1,000 g to 150,000 g, is between about 1,000 to 100,000 g, is between about 2,000 g to 200,000 g, is between about 2,000 g to 150,000 g, is between about 2,000 to 100,000 g, is between about 3,000 g to 200,000 g, is between about 3,000 g to 150,000 g, is between about 3,000 to 100,000 g, is between about 4,000 g to 200,000 g, is between about 4,000 g to 150,000 g, is between about 4,000 to 100,000 g, is between about 5,000 g to 200,000 g, is between about 5,000 g to 150,000 g, or is between about 5,000 to 100,000 g. In particular embodiments, the centrifugation of the algae extract, after the bursting of the microalgae cells with the high-pressure homogenization, is between about 4,000 g to 100,000 g.

In some embodiments, the centrifugation of the algae extract, after the bursting of the microalgae cells with the high-pressure homogenization, is at a temperature at between about 1° C. to 15° C., between about 1° C. to 14° C., between about 2° C. to 15° C., between about 2° C. to 14° C., between about 3° C. to 15° C., between about 3° C. to 14° C., between about 4° C. to 15° C., between about 4° C. to 14° C., between about 5° C. to 15° C., or between about 5° C. to 14° C. In particular embodiments, the centrifugation of the algae extract, after the bursting of the microalgae cells with the high-pressure homogenization, is at a temperature at between about 4° C. to 14° C.

Occasionally, after the high-pressure homogenization, precipitation of the lysed microalgae cells may occur. Dissolution of the precipitated microalgae cells is necessary for recovery of the microalgae components as there can be a significant amount of protein precipitation. Moreover, the soluble proteins present in the soluble fraction may not be stable for long, resulting in loss of the soluble proteins as well when massive precipitation occurs.

Alternative precipitation methods can include the use of heat or the boiling of microalgae, followed by an extraction. See Choi et al., Scientific Reports (2021), 11 (1): 1-11 and Ng et al. Front Bioeng. Biotechnol. (2020), 8. While these methods, may result in high protein concentrations, protein yield is not ideal (some protein is still not) and specific proteins (e.g., less soluble proteins) may not be recovered. The extraction method described herein recovers unstable proteins, retaining a more extensive variety of microalgae derived proteins and molecules that can be used to enrich cell media.

In some embodiments, the extraction of the microalgae derived components further comprises the addition of a detergent solution to the homogenized microalgae cells. In some embodiments, the detergent solution comprises a non-ionic surfactants. Non-ionic surfactants can include, but are not limited to, fatty alcohol ethoxylates, alkyl phenols, alkylphenol ethoxylates, ethoxylates, fatty acid ethoxylates, fatty acid alkoxylates, fatty acid alkanolamide ethoxylates, polyoxyethylene, polyols, polysorbates, sorbitans, fatty alcohol polyglycol ethers, PEGs, alkyl polyglucosides (APG) and laureth-(number) s. In some embodiments, the detergent solution comprises a surfactant that includes but is not limited to Triton X-100, CHAPS, NP-40, octyl thioglucoside, octyl glucoside, and dodecyl maltoside. In some embodiments, the detergent solution comprises a surfactant that is selected from the group consisting of Triton X-100, CHAPS, and NP-40. In some embodiments, the detergent solution comprises a surfactant comprising CHAPS. In some embodiments, the detergent solution comprises a surfactant comprising NP-40. In some embodiments, the detergent solution comprises a surfactant comprising Triton X-100.

In some embodiments, the detergent solution comprises between about 0.1% to 1.5%, between about 0.1% to 1.0%, between about 0.1% to 0.5%, between about 0.5% to 1.5%, between about 0.5% to 1.0%, or between about 1.0% to 1.5% of the surfactant comprising Triton X-100 in a phosphate buffered saline solution, each inclusive. In some embodiments, the detergent solution comprises between about 0.4% to 1.0% of the surfactant comprising Triton X-100 in a phosphate buffered saline solution. The addition of Triton X-100 is an improvement from previous methods because Triton X-100 allows for the recovery of a more extensive variety of proteins and molecules or a higher yield of proteins and molecules.

In some embodiments, the detergent solution used for between about 1 to 96 hours, 1 to 72 hours, 1 to 48 hours, 1 to 24 hours, 24 to 96 hours, 24 to 72 hours, 24 to 48 hours, 48 to 96 hours, 48 to 72 hour or 72 to 96 hours, each inclusive. In particular embodiments, the detergent solution is used for 24 to 72 hours, inclusive. In some embodiments, the detergent solution is used at or at about 2° C. to 12° C., at or at about 2° C. to 10° C., at or at about 4° C. to 12° C., or at or at about 4° C. to 10° C., each inclusive. In particular embodiments, the detergent solution is used at or at about 4° C. to 10° C., with or without shaking. In particular embodiments, the detergent solution is used for 24 to 72 hours, inclusive, at or at about 4° C. to 10° C., inclusive with or without shaking.

In some embodiments, the detergent solution comprising the Triton X-100 in a phosphate buffered saline solution is used for between about 1 to 96 hours, 1 to 72 hours, 1 to 48 hours, 1 to 24 hours, 24 to 96 hours, 24 to 72 hours, 24 to 48 hours, 48 to 96 hours, 48 to 72 hour or 72 to 96 hours, each inclusive. In particular embodiments, the detergent solution comprising the Triton X-100 in a phosphate buffered saline solution is used for 24 to 72 hours, inclusive. In some embodiments, the detergent solution comprising the Triton X-100 in a phosphate buffered saline solution is used at or at about 2° C. to 12° C., at or at about 2° C. to 10° C., at or at about 4° C. to 12° C., or at or at about 4° C. to 10° C., each inclusive. In particular embodiments, the detergent solution comprising the Triton X-100 in a phosphate buffered saline solution is used at or at about 4° C. to 10° C., with or without shaking. In particular embodiments, the detergent solution comprising the Triton X-100 in a phosphate buffered saline solution is used for 24 to 72 hours, inclusive, at or at about 4° C. to 10° C., inclusive with or without shaking.

In some embodiments, after the addition of the detergent solution, the homogenized, detergent-treated microalgae cells are centrifuged. In some embodiments, the homogenized, detergent-treated microalgae cells are centrifuged at between about 1000 g to 20,000 g, between about 1000 to 15,000 g, between about 1 to 10,000 g, between about 1 to 5,000 g, between about 5,000 to 20,000 g, between about 5,000 to 15,000 g, between about 5,000 to 10,000 g, between about 10,000 to 20,000 g, between about 10,000 to 15,000 g, or between about 15,000 to 20,000 g, each inclusive. In some embodiments, the homogenized, detergent-treated microalgae cells are centrifuged at between about 5,000 g to 15,000 g.

In some embodiments, the homogenized, detergent-treated microalgae cells are centrifuged at between about 1° C. to 5° C., between about 1° C. to 10° C. between about 1° C. to 15° C., between about 5° C. to 10° C., between about 5° C. to 15° C. or between about 10° C. to 15° C., each inclusive. In some embodiments, the homogenized, detergent-treated microalgae cells are centrifuged at between about 1° C. to 5° C.° C., inclusive.

In some embodiments, the centrifugation of the algae extract, after treatment with the detergent solution, is between about 1,000 g to 200,000 g, is between about 1,000 g to 150,000 g, is between about 1,000 to 100,000 g, is between about 2,000 g to 200,000 g, is between about 2,000 g to 150,000 g, is between about 2,000 to 100,000 g, is between about 3,000 g to 200,000 g, is between about 3,000 g to 150,000 g, is between about 3,000 to 100,000 g, is between about 4,000 g to 200,000 g, is between about 4,000 g to 150,000 g, is between about 4,000 to 100,000 g, is between about 5,000 g to 200,000 g, is between about 5,000 g to 150,000 g, or is between about 5,000 to 100,000 g. In particular embodiments, the centrifugation of the algae extract, after treatment with the detergent solution, is between about 4,000 g to 100,000 g.

In some embodiments, the centrifugation of the algae extract, after the treatment with the detergent solution, is at a temperature at between about 1° C. to 15° C., between about 1° C. to 14° C., between about 2° C. to 15° C., between about 2° C. to 14° C., between about 3° C. to 15° C., between about 3° C. to 14° C., between about 4° C. to 15° C., between about 4° C. to 14° C., between about 5° C. to 15° C., or between about 5° C. to 14° C.

After centrifugation, the resulting supernatant containing the microalgae-derived proteins and other molecules to be recovered may be further concentrated by filtration. In particular embodiments, the resulting supernatant containing the microalgae-derived proteins and other molecules to be recovered is further concentrated by ultra-filtration. In some embodiments, the centrifuged supernatant is filtered. In some embodiments, the filtration comprises the use of a filter with a pore size between about 0.22 μm to 0.45 μm.

In some embodiments, after recovery of the resulting supernatant, the resulting supernatant containing the microalgae-derived proteins and other molecules is sterilized using gamma rays.

In some embodiments, the filtration decreases the chance of contamination. In some embodiments, the filtration decreases the chance of contamination by between about 20% to 100%, between about 20% to 80%, between about 20% to 60%, between about 20% to 40%, between about 40% to 100%, between about 40% to 80%, between about 40% to 60%, between about 60% to 100%, between about 60% to 80%, or between about 80% to 100%, each inclusive. In some embodiments, the filtration decreases the chance of contamination by between about 60% to 100%, inclusive. Decreasing the chance of contamination is advantageous because there is no need to further disinfect or sterilize the centrifuged supernatant containing the microalgae-derived proteins and other molecules for culture media enrichment.

In some embodiments, the centrifuged, filtered supernatant containing the microalgae-derived proteins and other molecules is refrigerated. The ability to refrigerate the filtered supernatant is advantageous because it demonstrates the filtered supernatant is stable and microalgae-derived proteins and other molecules will not degrade. It is also advantageous because the filtered supernatant containing the microalgae-derived proteins and other molecules can be saved for later use. In some embodiments, the centrifuged, filtered supernatant is stored at between about 0° C. to 6° C., between about 0° C. to 4° C., between about 2° C. to 6° C., between about 2° C. to 4° C. or between about 4° C. to 6° C., each inclusive.

In some embodiments, prior to refrigeration, the centrifuged, filter supernatant containing the microalgae-derived proteins and other molecules is sterilized using gamma rays.

In some embodiments, the centrifuged, filtered supernatant is evaluated. In some embodiments, the centrifuged, filtered supernatant is evaluated at an optical density (OD) at 280 nm. The final concentration of recovered microalgae-derived proteins and other molecules, at an OD of 280 nm, should be >50 mg/ml.

In some embodiments, the centrifuged, filtered supernatant may be diluted in cell media. In some embodiments, the centrifuged, filtered supernatant is diluted in cell media. In some embodiments, the centrifuged, filtered supernatant is diluted in cell media to between about 5 mg/ml to 20 mg/ml, between about 5 mg/ml to 15 mg/ml, between about 5 mg/ml to 10 mg/ml, between about 10 mg/ml to 20 mg/ml, between about 10 mg/ml to 15 mg/ml, or between about 15 mg/ml to 20 mg/ml, each inclusive. In some embodiments, the centrifuged, filtered supernatant is diluted in cell media to between about 5 mg/ml to 15 mg/ml, inclusive. In some embodiments, the centrifuged, filtered supernatant is diluted in cell media to or to about 10 mg/ml.

In certain embodiments, the lysing of the microalgae cells comprises homogenizing the microalgae cells in water in accordance with the variations described herein, in combination with one more additional suitable methods and techniques known in the art, including, for example, sonication, chemical lysing, or heating. In alternative embodiments, the lysing of the microalgae cells may be performed by other suitable methods and techniques known in the art, including, for example, sonication, chemical lysing, or heating.

### V. Reformulated/Enriched Culture Media

In some embodiments, the enriched growth medium comprises available medium ingredients. In some embodiments the enriched growth medium comprises microalgae-derived components.

In some embodiments, the growth medium consists of the microalga-derived material. In some embodiments, the growth medium consists essentially of the microalga-derived material.

As used herein, the term “consisting essentially or’ denotes that a given compound or substance constitutes the vast majority of the active ingredient's portion or fraction of the composition.

In some embodiments, consisting essentially of means that the microalga-derived material constitutes at least 95%, at least 98%, at least 99%, or at least 99.9% by weight, of the active ingredient(s) of the growth medium provided to the cell, according to the herein disclosed method, or any value and range therebetween. Each possibility represents a separate embodiment of the invention.

In some embodiments, the enriched culture media uses a non-recycled culture media as the basal media. In some embodiments, the enriched culture media uses the recycled culture media produced by the methods of described herein as the basal media.

In some embodiments, the centrifuged, filtered supernatant, containing the microalgae-derived proteins and other molecules is added to the recycled media produced by the methods of recycling described in Section III. Even though the microalgae consumes the ammonia present in the spent media, the recycled, spent media as described in Section III contains a surplus of sugars such as glucose and pyruvate that can be utilized by the cells. In particular embodiments, the recycled, spent media still contains 80-100%, 80-95%, 80-90%, 80-85%, 85-100%, 85-95%, 85-90%, 90-100%, 90-95 or 95-100%, each inclusive, of its initial amount of pyruvate. In particular embodiments, the recycled spent media still contains 85-95%, inclusive, of its initial amount of pyruvate. In particular embodiments, the recycled spent media still contains about 91% of its initial amount of pyruvate.

The recycled, spent media may first be diluted in cell media, as described in Section IV, prior to be added as a media supplementation. In certain embodiments, the extract is added as a media supplementation between about 0.005 mg/ml to 0.1 mg/ml, 0.005 mg/ml to 0.05 mg/ml, 0.005 mg/ml to 0.01 mg/ml, 0.01 mg/ml to 0.1 mg/ml, 0.01 mg/ml to 0.05 mg/ml, or 0.05 mg/ml to 0.1 mg/ml, each inclusive. In particular embodiments, the extract is added as a media supplementation between about 0.005 mg/ml to 0.05 mg/ml, inclusive. In particular embodiments, the extract is added as a media supplementation at or at about 0.1 mg/ml.

In some embodiments, the extract is added as a media supplementation without fetal bovine serum. In some embodiments, the extract is added as a media supplementation with fetal bovine serum.

The recycled, spent media may require an adjustment prior to being re-used as culture media in the cultivation methods described in Section I. The recycled media is analyzed for nutrient content and components may be added, then it is filtered and added back to the animal cell culture. Adjustments can include nutrients supplementations such as supplementation of glutamine, glucose and/or the microalgae-derived proteins and other molecules. In some embodiments, embodiments, the reformulated media is supplemented with glutamine.

In some embodiments, glutamine is depleted or nearly depleted after recycling. In other variations, at least a portion is depleted. In certain variations, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the glutamine is depleted.

In some embodiments, the recycled, spent media may be depleted with glucose. In particular embodiments, the recycled spent media still contains 30-70%, 30-60%, 30-50%, 30-40%, 40-70%, 40-60%, 40-50%, 50-70%, 50-60%, or 60-70%, each inclusive, of its initial amount of glucose. In particular embodiments, the recycled spent media still contains 50-60%, inclusive, of its initial amount of glucose. In particular embodiments, the recycled spent media still contains about 56% of its initial amount of glucose. In particular embodiments, the reformulated media is supplemented with glucose.

In particular embodiments, for preparations of algal extract-based growth media for primary cells and cell lines culturing, the following ingredients are reduced or removed completely: FBS, HMM, taurine, glucosamine, and glutamine. In particular embodiments, depleted media are then supplemented to contain 0.5-50% of one or more algal extract combinations.

In some embodiments, the algae extract can be used to enrich cell growth medium. In certain embodiments, the cell growth medium can be a new cell growth medium. In certain embodiments, the cell growth medium is not the recycled media described herein. The cell growth medium can be formulated from salts and the algae extract can be used to enrich the cell growth media. The algae extract can enrich the medium with at least one compound, including but not limited to: fatty acids, antioxidants, proteins, pigment molecules, amino acids, vitamins, soluble molecules, and volatile molecules.

In certain embodiments, for the culturing of molluscan primary cells, two media formulations are used. The first is seawater media-SSW supplemented with 10% Leibovitz L-15, 10% fetal bovine serum (FBS), 10 mM HEPES buffer, and antibiotics IX (as mentioned in isolation section), pH 7.3-7.35, with or without supplementation of 12 mM calcium, magnesium, and sulfur cations. The second is artificial seawater media-Standard Leibovitz medium L-15 containing high concentration of amino acids, supplemented with or without one or more of the following: hemolymph of marine mollusks (HMM), NaCl (18.05 g/1), KCl (0.29 g/1), CaCh·2H2O (1.205 g/l), MgCh·6H2O (5.481 g/l), MgSO4·7H2O (4.28 g/l), taurine (25 mg/l), glucosamine (50 mg/l), glutamine (100 mg/l), FBS (2%) and gentamicin (40 mg/I). pH is adjusted to 7.6-7.8.

For both media types, osmolality is adjusted to 1,060-1,100 mOsm with NaCl and the medium is filter sterilized before use.

In some embodiments, the algae extract can be used to enrich the recycled media described herein. The algae extract can enrich the recycled media described herein with at least one compound, including but not limited to: fatty acids, antioxidants, proteins, pigment molecules, amino acids, vitamins, soluble molecules, and volatile molecules.

The enriched media, resulting from either enriching a new cell growth medium or the recycled medium described herein, is enriched by at least one compound, including but not limited to: fatty acids, antioxidants, proteins, pigment molecules, amino acids, vitamins, soluble molecules, and volatile molecules. In some embodiments, the enriched media, resulting from either enriching a new cell growth medium or the recycled medium described herein, comprises at least one compound selected from the group consisting of: fatty acids, antioxidants, proteins, pigment molecules, vitamins, soluble molecules, and volatile molecules. In particular embodiments, the enriched media is enriched with vitamins, such as, but not limited to, vitamin B2 (riboflavin).

In some embodiments, the algae extract enriches the media by fatty acids. In some embodiments, the enriched media comprises fatty acids. Incorporating a fatty acid into enriched media is advantageous because it may imbue the cells with the distinct and unique nutritional profile of the desired seafood product. In some embodiments, the fatty acid can include but is not limited to lauric acid, myristic acid, palmitic acid, stearidonic acid, oleic acid, elaidic acid, linoleic acid, α-linoleic acid, γ-linoleic acid, dihomo-γ-linoleic acid, arachidonic acid, tetracosapentaenoic acid, tetracosahexaenoic acid, eicosapentaenoic acid, eicosatetraenoic acid, docosatetraenoic acid, docosapentaenoic acid, and docosahexaenoic acid. In some embodiments, the fatty acid is selected from the group consisting of α-linoleic acid, eicosapentaenoic acid, docosatetraenoic acid, docosapentaenoic acid, docosahexaenoic acid, and tetracosahexaenoic acid. In some embodiments, the fatty acid is α-linoleic acid. In some embodiments, the fatty acid is eicosapentaenoic acid. In some embodiments, the fatty acid is docosahexaenoic acid. In some embodiments, the fatty acid is tetracosahexaenoic acid.

In some embodiments, algae extract enriches the media by antioxidants. In some embodiments, the enriched media comprises antioxidants. Incorporating an antioxidant into enriched media is advantageous because it may imbue the cells with the distinct and unique nutritional profile of the desired seafood product. The addition of antioxidants may also reduce the risk of many diseases. In some embodiments, the antioxidant can include but is not limited to bioflavonoids, carotenoids, terpenoids, vitamin C, and vitamin E. In some embodiments, the bioflavonoid is selected from the group consisting of flavonoids, flavanols, flavones, catechins, and anthocyanins. In some embodiments. The carotenoid is selected from the group consisting of alpha-carotene, beta-carotene, lutein, lycopene, and zeaxanthin. In some embodiments, the terpenoid is selected from the group consisting of isoprenoids and terpenes. In some embodiments, vitamin C is selected from the group consisting of ascorbic acid and ascorbate. In some embodiments, vitamin E is selected from the group consisting of tocopherols and tocotrienols. In some embodiments, the antioxidant is a bioflavonoid. In some embodiments, the antioxidant is a carotenoid. In some embodiments, the antioxidant is a terpenoid. In some embodiments, the antioxidant is vitamin C. In some embodiments, the antioxidant is vitamin E. In some embodiments, the antioxidant is alpha-carotene. In some embodiments, the antioxidant is beta-carotene. In some embodiments, the antioxidant is lutein. In some embodiments, the antioxidant is lycopene. In some embodiments, the antioxidant is zeaxanthin.

In some embodiments, the algae extract enriches the media by proteins. In some embodiments, the enriched media comprises proteins. Proteins in the media may be beneficial as carrier proteins, allowing and facilitating the cells to consume different molecules from the media, they also act as signal molecules for proliferation, help cells to attach one another or adhere and by so increase survival, stabilize osmolarity and more. In some variations, suitable proteins added to the media formulation are recombinant proteins or algae-derived proteins. In some variations, the proteins described here will not be derived from glans or animals.

It should be understood that, in some variations, the nutritional profile of the cells may be affected by amino acids, minerals and vitamins in the media.

In some embodiments, the algae extract enriches the media by amino acids. In some embodiments, the enriched media comprises amino acids. Incorporating amino acids into enriched media is advantageous because it may imbue the cells with the amino acids, adding more nutritional value to the desired seafood product. In some embodiments, the amino acid can be, but is not limited to, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some embodiments, the amino acid can be, but is not limited to, methionine, threonine, histidine, valine, phenylalanine, isoleucine, tryptophan, lysine and leucine.

In some embodiments, the algae extract enriches the media by vitamins. In some embodiments, the enriched media comprises vitamins. Incorporating vitamins into enriched media is advantageous because it may imbue the cells with the vitamins, adding more nutritional value to the desired seafood product. In some embodiments, the vitamin can be, but is not limited to, vitamin A, vitamin B1 (thiamin), vitamin B2 (riboflavin), vitamin B3 (niacin), vitamin B6 (pyridoxine), vitamin B7 (biotin), vitamin B9 (folate), vitamin B12, vitamin C, vitamin D, vitamin E, vitamin K.

In some embodiments, the enriched media comprises pigment molecules. The ability to incorporate a pigment molecule offers an advantage because the color and appearance of the resulting product can be altered or manipulated to achieve a desired or specific look. In some embodiments, the pigment molecule can include but is not limited to anthocyanins, betalins, carotenoids, flavonoids, chlorophyll, and diarylheptanoids. In some embodiments, the pigment molecule is an anthocyanin. In some embodiments, the anthocyanin is selected from the group consisting of aurantinidin, cyanidin, delphinidin, europinidin, pelargonidin, malvidin, peonidin, petunidin, and rosinidin. In some embodiments, the anthocyanin is aurantinidin. In some embodiments, the anthocyanin is cyanidin. In some embodiments, the anthocyanin is pelargonidin. In some embodiments, the anthocyanin is rosinidin. In some embodiments, the pigment molecule is a betalin. In some embodiments the betalin is selected from the group consisting of betacyanins and betaxanthins. In some embodiments the betalin is a betacyanin. In some embodiments the betalin is a betaxanthin. In some embodiments, the pigment molecule is a carotenoid. In some embodiments, the carotenoid can include but is not limited to Lycopersene, Phytofluene, Lycopene, Hexahydrolycopene, Torulene, α-Zeacarotene, α-Carotene, β-Carotene, γ-Carotene, δ-Carotene, ε-Carotene, ζ-Carotene, Alloxanthin, Bacterioruberin, Cynthiaxanthin, Pectenoxanthin, Cryptomonaxanthin, Crustaxanthin, Gazaniaxanthin, OH-Chlorobactene, Loroxanthin, Lutein, Lycoxanthin, Rhodopin, Rhodopinol, Saproxanthin, Zeaxanthin, Oscillaxanthin, Phleixanthophyll, Rhodovibrin, Spheroidene, Diadinoxanthin, Luteoxanthin, Mutatoxanthin, Citroxanthin, Zeaxanthin furanoxide, Neochrome, Foliachrome, Trollichrome, Vaucheriaxanthin, Rhodopinal, Warmingone, Torularhodinaldehyde, Torularhodin, Torularhodin methyl ester, Astacene, Astaxanthin, Canthaxanthin, Capsanthin, Capsorubin, Cryptocapsin, 2,2′-Diketospirilloxanthin, Echinenone, 3′-Hydroxyechinenone, Flexixanthin, 3-OH-Canthaxanthin, Hydroxyspheriodenone, Okenone, Pectenolone, Phoeniconone, Phoenicopterone β,ε-caroten-4-one, Rubixanthone, Siphonaxanthin, Astacein, Fucoxanthin, Isofucoxanthin, Physalien, Siphonein, β-Apo-2′-carotenal 3′,4′-Didehydro-2′-apo-b-caroten-2′-al, Apo-2-lycopenal, Apo-6′-lycopenal 6′-Apo-y-caroten-6′-al, Azafrinaldehyde, Bixin, Citranaxanthin, Crocetin, Crocetinsemialdehyde, Crocin Digentiobiosyl, Hopkinsiaxanthin, Methyl apo-6′-lycopenoate, Paracentrone, Sintaxanthin, Actinioerythrin, β-Carotenone, Peridinin, Pyrrhoxanthininol, Semi-α-carotenone, Semi-β-carotenone, Triphasiaxanthin, Eschscholtzxanthin, Eschscholtzxanthone, Rhodoxanthin, Tangeraxanthin, Nonaprenoxanthin, Decaprenoxanthin, and Bacterioruberin. In some embodiments, the pigment molecule is a flavonoid. In some embodiments, the flavonoid is selected from the group consisting flavanols, flavones, isoflavonoids, and neoflavonoids. In some embodiments, the pigment molecule is a diarylheptanoid. In some embodiments, the diarylheptanoid is selected from the group consisting of curcuminoids and cyclic diarylheptanoids. In some embodiments, the diarylheptanoid is a curcuminoid. In some embodiments, the diarylheptanoid is a cyclic diarylheptanoid.

In some embodiments, the enriched media comprises volatile molecules. The ability to incorporate a volatile molecule offers an advantage because the smell of the resulting product can be altered or manipulated to achieve a desired or specific smell. In some embodiments, the volatile molecule is selected from the group consisting of alcohols, terpenes, aldehydes, esters, furans, and pyrazines. In some embodiments, the volatile molecule is an alcohol. In some embodiments, the alcohol is hexanol. In some embodiments, the volatile molecule is a terpene. In some embodiments, the terpene is selected from the group consisting of myrcene, pinene, limonene, and linalool. In some embodiments, the volatile molecule is an aldehyde. In some embodiments, the aldehyde is hexanal or octanal. In some embodiments, the volatile molecule is an ester. In some embodiments, the ester is ethyl octanoate or ethyl hexanoate. In some embodiments, the volatile molecule is a furan. In some embodiments, the furan is furan, furfural, or furaneol. In some embodiments, the volatile molecule is a pyrazine. In some embodiments, the pyrazine is 2,5-dimethylpyrazine, ethylpyrazine, or methylpyrazine.

In some embodiments, when compared to that of the recycled media, the concentration of fatty acid of the enriched media is increased by between about 10 to 100 percent, between about 10 to 70 percent, 10 to 40 percent, 40 to 100 percent, 40 to 70 percent, or 70 to 100 percent, each inclusive. In some embodiments, when compared to that of the recycled media, the concentration of fatty acid of the enriched media is increased by between about 10 to 100 percent, inclusive. In some embodiments, when compared to that of the recycled media, the concentration of fatty acid of the enriched media is increased by between about 10 to 70 percent, inclusive. In some embodiments, when compared to that of the recycled media, the concentration of fatty acid of the enriched media is increased by between about 10 to 40 percent, inclusive. In some embodiments, when compared to that of the recycled media, the concentration of fatty acid of the enriched media is increased by between about 40 to 100 percent, inclusive. In some embodiments, when compared to that of the recycled media, the concentration of fatty acid of the enriched media is increased by between about 40 to 70 percent, inclusive. In some embodiments, when compared to that of the recycled media, the concentration of fatty acid of the enriched media is increased by between about 70 to 100 percent, inclusive.

In some embodiments, when compared to that of the recycled media, the concentration of antioxidants of the enriched media is increased by between about 10 to 100 percent, between about 10 to 70 percent, 10 to 40 percent, 40 to 100 percent, 40 to 70 percent, or 70 to 100 percent, each inclusive. In some embodiments, when compared to that of the recycled media, the concentration of antioxidants of the enriched media is increased by between about 10 to 100 percent, inclusive. In some embodiments, when compared to that of the recycled media, the concentration of antioxidants of the enriched media is increased by between about 10 to 70 percent, inclusive. In some embodiments, when compared to that of the recycled media, the concentration of antioxidants of the enriched media is increased by between about 10 to 40 percent, inclusive. In some embodiments, when compared to that of the recycled media, the concentration of antioxidants of the enriched media is increased by between about 40 to 100 percent, inclusive. In some embodiments, when compared to that of the recycled media, the concentration of antioxidants of the enriched media is increased by between about 40 to 70 percent, inclusive. In some embodiments, when compared to that of the recycled media, the concentration of antioxidants of the enriched media is increased by between about 70 to 100 percent, inclusive.

In some embodiments, when compared to that of the recycled media, the concentration of proteins of the enriched media is increased by between about 10 to 100 percent, between about 10 to 70 percent, 10 to 40 percent, 40 to 100 percent, 40 to 70 percent, or 70 to 100 percent, each inclusive. In some embodiments, when compared to that of the recycled media, the concentration of proteins of the enriched media is increased by between about 10 to 100 percent, inclusive. In some embodiments, when compared to that of the recycled media, the concentration of proteins of the enriched media is increased by between about 10 to 70 percent, inclusive. In some embodiments, when compared to that of the recycled media, the concentration of proteins of the enriched media is increased by between about 10 to 40 percent, inclusive. In some embodiments, when compared to that of the recycled media, the concentration of proteins of the enriched media is increased by between about 40 to 100 percent, inclusive. In some embodiments, when compared to that of the recycled media, the concentration of proteins of the enriched media is increased by between about 40 to 70 percent, inclusive. In some embodiments, when compared to that of the recycled media, the concentration of proteins of the enriched media is increased by between about 70 to 100 percent, inclusive.

In some embodiments, when compared to that of the recycled media, the concentration of pigment molecule of the enriched media is increased by between about 10 to 100 percent, between about 10 to 70 percent, 10 to 40 percent, 40 to 100 percent, 40 to 70 percent, or 70 to 100 percent, each inclusive. In some embodiments, when compared to that of the recycled media, the concentration of pigment molecule of the enriched media is increased by between about 10 to 100 percent, inclusive. In some embodiments, when compared to that of the recycled media, the concentration of pigment molecule of the enriched media is increased by between about 10 to 70 percent, inclusive. In some embodiments, when compared to that of the recycled media, the concentration of pigment molecule of the enriched media is increased by between about 10 to 40 percent, inclusive. In some embodiments, when compared to that of the recycled media, the concentration of pigment molecule of the enriched media is increased by between about 40 to 100 percent, inclusive. In some embodiments, when compared to that of the recycled media, the concentration of pigment molecule of the enriched media is increased by between about 40 to 70 percent, inclusive. In some embodiments, when compared to that of the recycled media, the concentration of pigment molecule of the enriched media is increased by between about 70 to 100 percent, inclusive.

In some embodiments, when compared to that of the recycled media, the concentration of volatile molecule of the enriched media is increased by between about 10 to 100 percent, between about 10 to 70 percent, 10 to 40 percent, 40 to 100 percent, 40 to 70 percent, or 70 to 100 percent, each inclusive. In some embodiments, when compared to that of the recycled media, the concentration of volatile molecule of the enriched media is increased by between about 10 to 100 percent, inclusive. In some embodiments, when compared to that of the recycled media, the concentration of volatile molecule of the enriched media is increased by between about 10 to 70 percent, inclusive. In some embodiments, when compared to that of the recycled media, the concentration of volatile molecule of the enriched media is increased by between about 10 to 40 percent, inclusive. In some embodiments, when compared to that of the recycled media, the concentration of volatile molecule of the enriched media is increased by between about 40 to 100 percent, inclusive. In some embodiments, when compared to that of the recycled media, the concentration of volatile molecule of the enriched media is increased by between about 40 to 70 percent, inclusive. In some embodiments, when compared to that of the recycled media, the concentration of volatile molecule of the enriched media is increased by between about 70 to 100 percent, inclusive.

### VI. Resulting Food Compositions

In some embodiments, the cultured cell is essentially similar to a tissue from which it is derived in at least one characteristic being selected from: fatty acids, types and content, carotenoids, protein profile and content, genetic or genomic composition, nutritional profile, or any combination thereof. In some embodiments, nutritional profile refers to the content, profile, or both of: ash, water, proteins, fats, carbohydrates, or any combination thereof.

Exemplary cellular characterization assays can include, but are not limited to: growth assay, fatty acid composition analysis assay, volatile compounds analysis assay and nutritional value analysis assay. For instance, in a growth assay, cellular viability and proliferation rates can be assessed either by the XTT assay (Biological Industries), by trypan blue exclusion test adapted to marine environment, or by daily dissociation with trypsin followed by cell numbers and viability assessment by an automated cell counter.

According to some embodiments, there is provided a composition comprising a cell cultured according to the herein disclosed method, and an acceptable carrier.

In some embodiments, the composition is being an edible composition. In some embodiments, the composition is a food product, food stuff, or any substrate or precursor being used in the preparation of same.

In some embodiments, the edible composition disclosed herein, is essentially similar to an edible part of an aquatic organism in at least one characteristic being selected from: taste, aroma, appearance, handling, mouthfeel, density, structure, texture, elasticity, springiness, coagulation, binding, leavening, aeration, foaming, creaminess, emulsification, or any combination thereof.

In some embodiments, the edible part comprises or consists of a muscle tissue.

In some embodiments, the edible composition is raw or cooked. In some variations, cooked encompasses any method of cooking which is available and commonly practices by a person of ordinary skill in the art, e.g., frying, backing, grilling, to name a few.

In some embodiments, an edible composition as disclosed herein in its raw form or state is essentially similar to an edible part of an aquatic organism in its raw form or state in at least one characteristic, as disclosed herein.

In some embodiments, an edible composition as disclosed herein in its cooked form or state is essentially similar to an edible part of an aquatic organism in its cooked form or state in at least one characteristic, as disclosed herein.

As used herein, being essentially similar refers to being at least 50%, at least 60%, at least 70%, at least 80%, at least 90%, at least 95%, at least 99%, or 100% identical, or any value and range therebetween. Each possibility represents a separate embodiment of the invention.

The carrier may comprise, in total, from about 0.1% to about 99.99999% by weight of the composition presented herein, such as the edible composition of the invention.

In some embodiments, the edible composition comprises a seafood alternative or analog. In some embodiments, the edible composition comprises a cultured seafood.

In some embodiments, the term “seafood” as used herein, comprises at least partially, a muscle tissue derived from an aquatic organism.

As used herein, the term “food product” refers to a material, a substance, or an additive, which can be used as food, or which can be added to food. Typically, the food product is any composition that an animal, preferably a mammal such as a human, may consume as part of its diet.

In some embodiments, an edible composition or a food product comprising same, according to the present invention, is characterized by similar properties of a corresponding seafood product.

Properties of seafood that can be tested include mechanical properties such as hardness, cohesiveness, brittleness, chewiness, gumminess, viscosity, elasticity, and adhesiveness. Additional properties can include moisture content and fat content. These properties can be described using terms such as “soft”, “firm” or “hard” describe hardness; “crumbly”, “crunchy”, “brittle”, “chewy”, “tender”, “tough”, “short”, “mealy”, “pasty”, or “gummy” to describe cohesiveness; “thin” or “viscous” to describe viscosity; “plastic” or “elastic” to describe elasticity; “sticky”, “tacky” or “gooey” to describe adhesiveness; “dry”, “moist”, “wet” or “watery” to describe moisture content; or “oily” or “greasy” to describe fat content.

In some embodiments, a biomass of cultivated cells is produced. In some embodiments, the biomass of cultivated cells is used as raw material for the construction of cultivated food products. In some embodiments, the cultivated food products can include but are not limited to fish and shellfish food products. In some embodiments, the cultivated food products are selected from the group consisting of: scallops, shrimps, crab cakes, and surimi products. In some embodiments, the cultivated food products are scallops. In some embodiments, the cultivated food products are shrimps. In some embodiments, the cultivated food products are crab cakes. In some embodiments, the cultivated food products are surimi products.

In some embodiments, the biomass of cultivated cells is washed from leftover growth media. In some embodiments, the biomass of cultivated cells is washed with new media, leftover media, or a non-media solution. In some embodiments, the biomass of cultivated cells is washed with new media. In some embodiments, the biomass of cultivated cells is washed with leftover media. In some embodiments, the biomass of cultivated cells is washed with a non-media solution.

In some embodiments, the biomass of cultivated cells can be washed no more than 6 times, 5 times, 4 times, 3 times, 2 times or 1 time. In some embodiments, the biomass of cultivated cells can be washed at least 1 time, at least 2 times, at least 3 times, at least 4 times, at least 5 times, or at least 6 times. In some embodiments, the biomass of cultivated cells can be washed between 1-6 times, 1-5 times, 1-4 times, 1-3 times, 1-2 time, 2-6 times, 2-5 times, 2-4 times, 2-3 times, 3-6 times, 3-5 times, 3-4 times, 4-6 times, 4-5 times or 5-6 times. In some embodiments, water is added to the biomass of cultivated cells.

In some embodiments, a hydrocolloid as a thickening or gelling agent is added to the biomass of cultivated cells. The ability to add a hydrocolloid to the biomass is advantageous because the shape and texture of the cultivated cells can be manipulated to achieve the desired shape and texture of the cultivated food product. In some embodiments, the hydrocolloid can include but is not limited to xanthan, carboxymethyl cellulose, methyl cellulose, hydroxypropylmethyl cellulose, gum Arabic, galactomannans, konjac maanan, gum tragacanth, modified starch, agar, κ-Carrageenan and í-carrageenan, low methoxy pectin, high methoxy pectin, gellan gum, and alginate. In some embodiments, the hydrocolloid is selected from the group consisting of xanthan, methyl cellulose, hydroxypropylmethyl cellulose, modified starch, agar, low methoxy pectin, and high methoxy pectin. In some embodiments, the hydrocolloid is xanthan. In some embodiments, the hydrocolloid is methyl cellulose. In some embodiments, the hydrocolloid is hydroxypropylmethyl cellulose. In some variations, the amount of hydrocolloid added is between 0.1 and 10%, or between 0.5% and 8%. In other variations, the amount of hydrocolloid added is between 0.1-10 g per 100 g of cells, between 0.5 and 10 g or cells, or between 0.5-8 g of cells.

In some embodiments, the biomass of cultivated cells is unmolded or molded. In some embodiments, the biomass of cultivated cells is unmolded. In some embodiments, the biomass of cultivated cells is molded. In some embodiments, the biomass of cultivated cells mixture is cold pressed to molds. In some embodiments, the biomass of cultivated cells is molded with the presence of fibers. In some embodiments, the biomass of cultivated cells is processed by cold extrusion to form the desired food product.

In some variations of the foregoing, prior to this step of cultivating cells, one or more additional ingredients may be added, e.g., after cells, water, and hydrocolloid. In certain variations, such one or more additional ingredients may include, for example, sugars, sugar alcohols, salts, phosphates, polyphosphates hydrocolloids. polysaccharides, fibers, flavorings, oils/fatty acids, coloring agents. In certain variations, the molding is performed after all the additives are included.

In other variations of the foregoing, functional fibers may added to the product mix for better texturizing, bite and mouthfeel. In certain variations, the molded storing temperatures are employed after all the additives are included.

In some embodiments, the molded biomass of cultivated cells are stored at between about −80° C. to 0° C., between about −80° C. to −20° C., between about −80° C. to −40° C., between about −80° C. to −60° C., between about −60° C. to 0° C., between about −60° C. to −20° C., between about −60° C. to −40° C., between about −40° C. to 0° C., between about −40° C. to −20° C., or between about −20° C. to 0° C. In some embodiments, the molded biomass of cultivated cells are stored at between about −80° C. to 0° C. In some embodiments, the molded biomass of cultivated cells are stored at between about −80° C. to −20° C. In some embodiments, the molded biomass of cultivated cells are stored at between about −80° C. to −40° C. In some embodiments, the molded biomass of cultivated cells are stored at between about −80° C. to −60° C. In some embodiments, the molded biomass of cultivated cells are stored at between about −60° C. to 0° C. In some embodiments, the molded biomass of cultivated cells are stored at between about −60° C. to −20° C. In some embodiments, the molded biomass of cultivated cells are stored at between about-60° C. to −40° C. In some embodiments, the molded biomass of cultivated cells are stored at between about −40° C. to 0° C. In some embodiments, the molded biomass of cultivated cells are stored at between about −40° C. to −20° C. In some embodiments, the molded biomass of cultivated cells are stored at between about −20° C. to 0° C.

In some embodiments, a flavoring is added to the biomass of cultivated cells. The ability to add a flavoring is advantageous because the flavor profile of the cultivated cells can be modified and the cultivated cells can closer mimic desired or specific seafood flavors. In some embodiments, the flavoring can include but is not limited to essential oil extracts, non-alcoholic flavoring extracts, true fruit flavors, imitation extracts, artificial extracts, and essences. In some embodiments, the flavoring is an artificial extract. In some embodiments, the flavoring is an imitation extract. In some embodiments, the flavoring is an essence.

In some embodiments, a coloring agent is added to the biomass of cultivated cells. The ability to add a coloring agent is advantageous because the color and appearance of the cultivated cells can be manipulated to achieve a desired look for the cultivated food product. In some embodiments, the coloring agent is selected from the group consisting of carotenoids, chlorophyllin, anthocyanins, and betanins. In some embodiments, the coloring agent is a carotenoid. In some embodiments, the coloring agent is chlorophyllin. In some embodiments, the coloring agent is an anthocyanin. In some embodiments, the coloring agent is a betanin.

In some embodiments, a fiber is added to the biomass of cultivated cells. The ability to add fiber is advantageous because fiber is important for the health of the digestive system and for lowering cholesterol. In some embodiments, the fiber is a natural fiber or an artificial fiber. In some embodiments, the fiber is a natural fiber. In some embodiments, the fiber is an artificial fiber.

In some embodiments, a plant-derived protein is added to the biomass of cultivated cells. The ability to add a plant-derived protein is beneficial because plant-based protein sources may help decrease the risk of developing chronic diseases including heart disease, diabetes, and cancers. In some embodiments, the plant-derived protein can include but is not limited to protein derived from beans, broccoli, chickpeas, greens, lentils, nut butter, nuts, seeds, peas, potatoes, quinoa, seaweed, soymilk, spinach, tempeh, tofu, and veggie patties. In some embodiments, the plant-derived protein is selected from the group consisting of protein derived from beans, chickpeas, greens, nuts, seeds, and tofu. In some embodiments, the plant-derived protein is selected from the group consisting of protein derived from beans. In some embodiments, the plant-derived protein is selected from the group consisting of protein derived from chickpeas. In some embodiments, the plant-derived protein is selected from the group consisting of protein derived from greens. In some embodiments, the plant-derived protein is selected from the group consisting of protein derived from nuts. In some embodiments, the plant-derived protein is selected from the group consisting of protein derived from seeds. In some embodiments, the plant-derived protein is selected from the group consisting of protein derived from tofu.

In some embodiments, the plant-derived protein is added at a concentration of between about 5 to 20 mM, between about 5 to 15 mM, between about 5 to 10 mM, between about 10 to 20 mM, between about 10 to 15 mM, between about 15 to 20 mM. In some embodiments, the plant-derived protein is added at a concentration of between about 5 to 20 mM. In some embodiments, the plant-derived protein is added at a concentration of between about 5 to 15 mM. In some embodiments, the plant-derived protein is added at a concentration of between about 5 to 10 mM. In some embodiments, the plant-derived protein is added at a concentration of between about 10 to 20 mM. In some embodiments, the plant-derived protein is added at a concentration of between about 10 to 15 mM. In some embodiments, the plant-derived protein is added at a concentration of between about 15 to 20 mM.

In some embodiments, the biomass of cultivated cells is raw or cooked. In some embodiments, the biomass of cultivated cells is raw. In some embodiments, the biomass of cultivated cells is cooked. In some embodiments, the biomass of cultivated cells is cooked at between about 40° C. to 80° C., between about 40° C. to 70° C., between about 40° C. to 60° C., between about 40° C. to 50° C., between about 50° C. to 80° C., between about 50° C. to 70° C., between about 50° C. to 60° C., between about 60° C. to 80° C., between about 60° C. to 70° C., between about 70° C. to 80° C.

### VI. Definitions

Any concentration ranges, percentage range, or ratio range recited herein are to be understood to include concentrations, percentages, or ratios of any integer within that range and fractions thereof, such as one tenth and one hundredth of an integer, unless otherwise indicated.

Any number range recited herein relating to any physical feature, such as polymer subunits, size, or thickness, are to be understood to include any integer within the recited range, unless otherwise indicated.

In the discussion unless otherwise stated, adjectives such as “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

### VI. Exemplary Embodiments

Among the provided embodiments are:

1. A method for cultivated food production, comprising:
(i) cultivating cells in a media, optionally wherein the media comprises enriched media;
(ii) withdrawing spent media resulting from the cultivating of cells in step (i);
(iii) proliferating algae in the presence of ammonia and/or ammonium from the spent media;
(iv) lysating the algae; and
(v) retrieving treated spent media to further cultivate cells in step (a), optionally, wherein at least a portion of the cultivated cells in step (i) are harvested at any time.

2. A method for cultivated food production, comprising:
(a) cultivating cells in a media, optionally wherein the media comprises enriched media;
(b) recycling spent media resulting from the cultivating of cells in step (a), wherein the recycling of the spent media is performed by microalgae; and
(c1) expanding the cells until a biomass of cultivated cells is reached, or
(c2) harvesting at least a portion of the cells and (d2) allowing remaining cells to expand upon repeating steps (a)-(c1) above.

3. The method of embodiment 2, wherein the media is enriched by microalgae derived components.

4. The method of any one of embodiments 1-3, wherein the cultivating of cells is a continuous culture, fed-batch culture, or batch culture.

5. The method of embodiment 4, wherein the cultivating of cells is continuous culture and cultivated cells are harvested to be used as raw material for food products.

6. The method of embodiment 4 or embodiment 5, wherein the harvesting of cultivated cells occurs between about every 8 hours to 24 hours, inclusive.

7. The method of any one of embodiments 1-6, wherein the harvesting of cultivated cells harvests between about 30 percent to 70 percent of the content of a bioreactor.

8. The method of any one of embodiments 1-7, wherein the harvesting of cultivated cells harvests at or about 50% of the content of a bioreactor.

9. The method of any one of embodiments 5-8, wherein the harvested, cultivated cells are separated from spent media.

10. The method of any one of embodiments 5-9, wherein the harvested, cultivated cells are used as fresh raw materials for cultivated food production.

11. The method of any one of embodiments 5-9, wherein the harvested, cultivated cells are further washed after the separation from the spent media in embodiment 9.

12. The method of embodiment 11, wherein the washing is performed with an isotonic solution and/or water.

13. The method of embodiment 11 or embodiment 12, wherein the washing occurs at least three times.

14. The method of any one of embodiments 11-13, wherein the washed cells are used as fresh raw materials for cultivated food production.

15. The method of any one of embodiments 10-14, wherein the unwashed or washed cells that are harvested are frozen.

16. The method of embodiment 15, wherein the cells are frozen with a cryoprotectant.

17. The method of embodiment 16, wherein the cryoprotectant is a food grade cryoprotectant.

18. The method of any one of embodiments 1-17, wherein the recycling of the spent media comprises transferring the spent media to a vessel containing microalgae.

19. The method of embodiment 18, wherein prior to the transferring of the spent media, the spent media is filtered.

20. The method of any one of embodiments 1-19, wherein the algae tank or bioreactor comprises an effective amount of microalgae to perform the recycling of the spent media.

21. The method of embodiment 20, wherein the effective amount of microalgae is between about 1×10³ to 1×10⁹ microalgae cells per milliliter of spent media.

22. The method of any one of embodiments 18-21, wherein the vessel containing microalgae is set up for growth under heterotrophic conditions.

23. The method of any one of embodiments 18-22, wherein the vessel containing microalgae is set up to preserve light sensitive elements in the spent media.

24. The method of any one of embodiments 1-23, wherein (i) the genus of microalgae is selected from the group consisting of: Chlorella, Porphyridiophyceae, Dunaliella, Nannochloropsis and Tetraselmis; or (ii) the genus of microalgae is Chlorococcum littorale; Chlorococcum submarinum; Porphyridium purpureum; Dunaliella salina; Dunaliella sp.; Chlorella vulgaris; Chlorella sp.; Arthrospira platensis; Euglena gracilis; Acutodesmus obliquus; Lobosphaera incisa; Tetraselmis striata; Tetraselmis sp.; Diacronema lutheri; Nannochloropsis oculate; Nannochloropsis sp.; Porphyridium purpureum (P. p); Isochrysis galbana (I. g); Phaeodactylum tricornutum (P. t); or Haematococcus pluvialis.

25. The method of any one of embodiments 1-24, wherein the microalgae reduces a concentration of at least one compound in the spent media selected from the group consisting of: ammonia, phosphate, glucose, glutamine and pyruvate.

26. The method of any one of embodiments 1-25, wherein the microalgae reduces a concentration of ammonia in the spent media.

27. The method of embodiment 25 or embodiment 26, wherein the concentration of ammonia is reduced by about 20% to 100%, inclusive.

28. The method of any one of embodiments 1-27, wherein the concentration of ammonia is reduced by about 80%.

29. The method of any one of embodiments 25-28, wherein the reduction of ammonia is completed within 1 to 96 hours, inclusive.

30. The method of any one of embodiments 25-29, wherein the microalgae further reduces a concentration of phosphate in the spent media.

31. The method of embodiment 25 or embodiment 30, wherein the concentration of phosphate is reduced by about 20% to 100%, inclusive.

32. The method of any one of embodiments 25 or 30-31, wherein the concentration of phosphate is reduced by about 30%.

33. A method of recycling spent media from the cultivation of cells for cultivated food production, comprising:
(a) separating the spent media from the cultured cells;
(b) introducing microalgae to the spent media;
(c) cultivating the microalgae under heterotrophic, autotrophic, and/or mixotrophic conditions, e.g., to recycle the spent media, reducing the concentration of ammonia; and
(d) separating the microalgae from the recycled media.

34. The method of embodiment 33, further comprising adjusting the recycled media by adding microalgae derived components, and optionally adjusting the recycled media by adding other nutrients (e.g., vitamins, amino acids).

35. The method of any one of embodiments 18-32, wherein the recycling of the spent media further comprises separating the microalgae from the recycled media.

36. The method of any one of embodiments 32-35, wherein the separation of the microalgae from the recycled media is performed by any suitable methods, optionally by centrifugation or by liquid/solid particle separation.

37. The method of embodiment 36, wherein the liquid/solid particle separation is performed by a rotary vacuum-drum filter.

38. The method of any one of embodiments 32-37, wherein the separated microalgae from the recycled media is separated into at least two parts.

39. The method of embodiment 38, wherein a first part of the at least two parts of the separated microalgae from the recycled media is placed back into the vessel containing microalgae for at least one other cycle.

40. The method of embodiment 38 or embodiment 39, wherein a second part of the at least two parts of the separated microalgae from the recycled media is frozen.

41. The method of any one of embodiments 38-40, wherein a second part of the at least two parts of the separated microalgae from the recycled media is extracted for the microalgae derived components.

42. The method of embodiment 41, wherein the extraction for the microalgae derived components comprises lysing the microalgae cells.

43. The method of embodiment 42, wherein the lysing of the microalgae cells comprises homogenizing the microalgae cells in isotonic solution and/or in water, sonication, chemical lysing, or heating.

44. The method of embodiment 43, wherein the homogenization of the microalgae cells comprises the use of high-pressure homogenization.

45. The method of embodiment 43 or embodiment 44, wherein the homogenization pressure is between about 5 to 40 kpsi.

46. The method of any one of embodiments 43-45, wherein the homogenized microalgae cells are centrifuged.

47. The method of any one of embodiments 43-45, wherein the extraction for the microalgae derived components further comprises the addition of a detergent solution to the homogenized microalgae cells.

48. The method of embodiment 47, wherein the detergent solution comprises a surfactant comprising a non-ionic surfactant, optionally wherein the non-ionic surfactant is Triton X-100.

49. The method of embodiment 48, wherein the detergent solution comprises 0.4 to 1.0% of the surfactant comprising Triton X-100 in a phosphate buffered saline solution.

50. The method of any one of embodiments 47-49, wherein after the addition of the detergent solution, the detergent treated microalgae cells are centrifuged.

51. The method of any one of embodiments 41-50, wherein the extraction for the microalgae derived components further comprises collecting a centrifuged supernatant.

52. The method of embodiment 51, wherein the centrifuged supernatant comprises soluble microalgae-derived components.

53. The method of embodiment 51, wherein the centrifuged supernatant comprising the microalgae derived components is filtered.

54. The method of embodiment 52 or embodiment 53, wherein the centrifuged, filtered supernatant comprising the microalgae derived components is refrigerated.

55. The method of any one of embodiments 1-54, wherein microalgae derived components of the enriched media in step (a) are derived from the same microalgae used for recycling the spent media in step (b).

56. The method of embodiment any one of embodiments 1-44, wherein the microalgae derived components of the enriched media in step (a) are derived from different microalgae used for recycling the spent media in step (b).

57. A method of extracting microalgae derived components, comprising:
(a) lysing microalgae cells, wherein the lysing is performed by high pressure homogenization;
(b) treating the lysed microalgae cells with a detergent to release soluble proteins;
(c) centrifugating the lysed microalgae cells treated with the detergent;
(d) collecting the supernatant; and
(f) concentrating the extracted microalgae derived components from the supernatant by centrifugation.

58. The method of embodiment 57, further comprising filtering the extracted microalgae derived components.

59. The method of embodiment 57 or embodiment 58, further comprising adding microalgae derived components to a cell medium to enrich the cell medium; and optionally adjusting the recycled media by adding other nutrients (e.g., vitamins, amino acids).

60. The method of any one of embodiments 53-58, wherein the centrifuged, filtered supernatant containing the microalgae derived components is added to the recycled media produced by the method of any one of embodiments 18-33 or 35-38 to formulate the enriched media used in any one of embodiments 1-59.

61. The method of embodiment 60, wherein the enriched media comprises microalgae derived components.

62. The method of any one of embodiments 1-61, wherein the cultivating of cells occurs in a stirred tank bioreactor.

63. The method of any one of embodiments 1-62, wherein the cells cultivated in step (a) of embodiment 1 are embryonic derived stem cells.

64. The method of embodiment 63, wherein the embryonic derived stem cells are derived from fish embryos.

65. The method of any one of embodiments 1-62, wherein the cells cultivated in step (a) of embodiment 1 are primary cells.

66. The method of embodiment 65, wherein the primary cells are derived from fish larval or fish adult tissues.

67. An extract containing microalgae derived components produced by the method of any one of embodiments 41-58.

68. An enriched culture media enriched by the extract of embodiment 67.

69. An enriched culture medium enriched by the method of or the algae extract produced by any one of embodiments 34 and 59-61.

70. The enriched culture medium of embodiment 68 or embodiment 69, wherein the enriched culture medium is used to cultivate cells.

71. A biomass of cultured cells produced by the method of any one of embodiments 1-17.

72. A biomass of cultured cells produced by the method of any one of embodiments 1-17 and cultivated with the enriched culture medium of any one of embodiments 67-69.

73. A cultivated seafood product produced by the method of any one of embodiments 1-72.

74. A method for culturing a cell being derived from an aquatic organism, the method comprising culturing said cell on a growth medium comprising microalga-derived material under conditions suitable for proliferation of said cell, thereby culturing the cell being derived from an aquatic organism.

75. The method of embodiment 74, wherein said microalga-derived material comprises: extract, homogenate, lysate, any fraction thereof, or any combination thereof, being derived from a microalga.

76. The method of embodiment 74 or 75, wherein said microalga is selected from the group consisting of: Chiarella vulgaris, Phaeodactylum tricornutum, Nannochloropsis oceanica, Jsochrysis galbana, Spirulina, Chlorococcum littorale, Arthrospira platensis, and any combination thereof.

77. The method of any one of embodiments 74 to 76, wherein said aquatic organism is an aquacultured organism.

78. The method of any one of embodiments 74 to 77, wherein said aquatic organism is selected from the group consisting of: a mollusk, a fish, and a crustacean.

79. The method of any one of embodiments 74 to 78, wherein said culturing is under a temperature ranging from 18-28° C.

80. The method of any one of embodiments 74 to 79, wherein said cell is selected from: a primary cell culture, an immortalized cell, or a cell line.

81. The method of any one of embodiments 74 to 80, wherein said cell is derived from a muscle tissue of said aquatic organism.

82. The method of any one of embodiments 74 to 81, wherein said growth medium consists of said microalga-derived material.

83. The method of any one of embodiments 74 to 82, wherein said cultured cell is essentially similar to a tissue from which it is derived in at least one characteristic being selected from the group consisting of: fatty acids profile and content, carotenoids profile and content, protein profile and content, and any combination thereof.

84. A composition comprising a cell cultured according to the method of any one of embodiments 74-83, and an acceptable carrier.

85. The composition of embodiment 84, being an edible composition.

86. The edible composition of embodiment 85, being essentially similar to an edible part of said aquatic organism in at least one characteristic being selected from the group consisting of: taste, aroma, appearance, handling, mouthfeel, density, structure, texture, elasticity, springiness, coagulation, binding, leavening, aeration, foaming, creaminess, emulsification, and any combination thereof.

87. The edible composition of embodiment 86, wherein said edible part comprises a muscle tissue of said aquatic organism.

88. The edible composition of embodiment 86 or 87, being raw or cooked.

89. A method for treating spent cell culture medium comprising contacting said spent cell culture medium with an effective amount of microalgae, thereby treating the spent cell culture medium.

90. The method of embodiment 89, wherein said spent cell cultured medium was used for culturing an animal cell.

91. The method of embodiment 90, wherein said animal cell is derived or obtained from any one of an invertebrate organism or a vertebrate organism.

92. The method of embodiment 91, wherein said invertebrate organism is selected from the group consisting of: mollusc, crustacean, and insect.

93. The method of embodiment 91, wherein said vertebrate organism is selected from the group consisting of: mammal, fish, and avian.

94. The method of embodiment 93, wherein said mammal is selected from the group consisting of: primate, bovine, rodent, and porcine.

95. The method of any one of embodiments 89 to 94, wherein said microalga is selected from the group consisting of: Chiarella vulgaris, Phaeadactylum tricarnutum, Nannachlarapsis aceanica, Isachrysis galbana, Spirulina, Chiarella vulgaris, and any combination thereof.

96. The method of any one of embodiments 89 to 95, wherein said treating comprises reducing the concentration of at least one compound selected from group consisting of: ammonia, CO2, phosphate, lactate, and any combination thereof, in said spent cell culture medium.

97. The method of any one of embodiments 89 to 96, further comprising a step comprising culturing a cell on said treated spent culture medium.

98. The method of any one of embodiments 89 to 97, wherein said contacting is for a time period ranging from 1 to 15 days.

99. The method of any one of embodiments 89 to 98, wherein said effective amount of microalgae ranges from 1×10⁴ to 1×10⁷ microalgae cells per ml of said spent culture medium.

100. A spent culture medium treated according to the method of any one of embodiments 89 to 99.

101. The spent culture medium of embodiment 100, characterized by being devoid of or comprising at least one compound selected from group consisting of: ammonia, CO2, phosphate, lactate, and any combination thereof, in an amount/concentration below a predetermined threshold.

### VII. Examples

The presently disclosed subject matter will be better understood by reference to the following Examples, which are provided as exemplary of the invention, and not by way of limitation.

### A. Example 1: Use of Algae to Clean Metabolites from Used Animal-Cell Growth Media

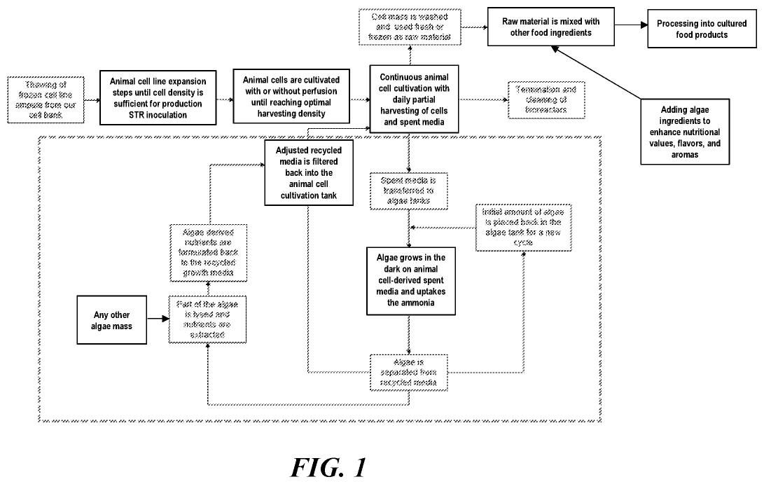

This example explores a process to recycle the spent media by cultivating microalgae that utilize ammonia and phosphate for reuse for animal cell growth. This processed in described inside the dashed box in FIG. 1.

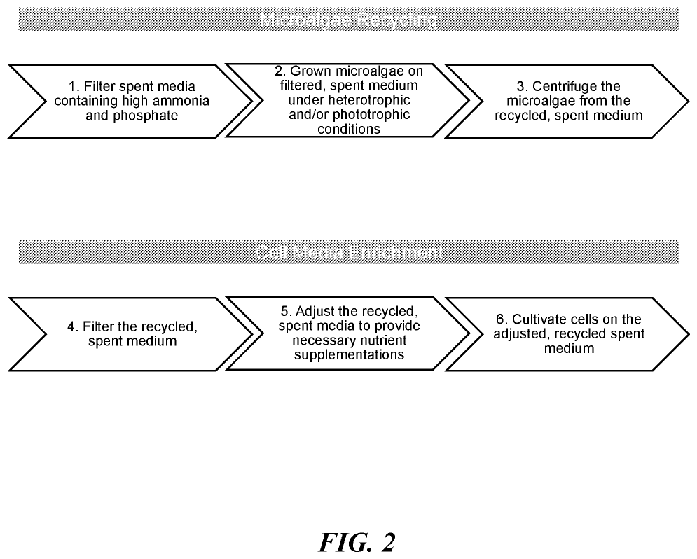

FIG. 2 describes the general process of using microalgae to recycle spent cell culture medium. The spent medium is filtered to remove cellular debris. Microalgae cells were grown in the filtered, spent medium under heterotrophic conditions. 1×10⁷ cells per ml were cultured for two days. After two days, the recycled medium was centrifuged to separate the microalgae from the recycled media. The recycled medium was filtered and assessed.

### Growth Under Heterotrophic Conditions

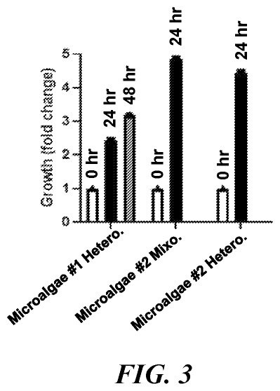

Two Chlorella species were shown to grow on Minimum Essential Medium (MEM) spent media (FIG. 3) under heterotrophic conditions. Chlorella vulgaris tripled its cell density within two days of cultivating heterotrophically on the spent media (starting from a density of 6×10⁷ cells/ml). The density of Chlorella sp. Was 4.5 times higher than from day 0 (starting from a density of 2×10⁷ cells/ml) after only one day of cultivating on spent media. In this experiment, the division rate was 4.98 hours. No significant change occurred between the mixotrophic and heterotrophic growth conditions of Chlorella sp. (denoted as “Microalgae #2 Mixo.” and “Microalgae #2 Hetero.” respectively). The growth of Chlorella sp. was similar between mixotrophic or heterotrophic growth, as demonstrated by FIG. 3. In heterotrophic conditions (denoted as “Microalgae #1 Hetero.”), Chlorella vulgaris utilized ammonia (NH3) from spent media more efficiently than in phototrophic conditions (data not shown).

### Ammonia Elimination

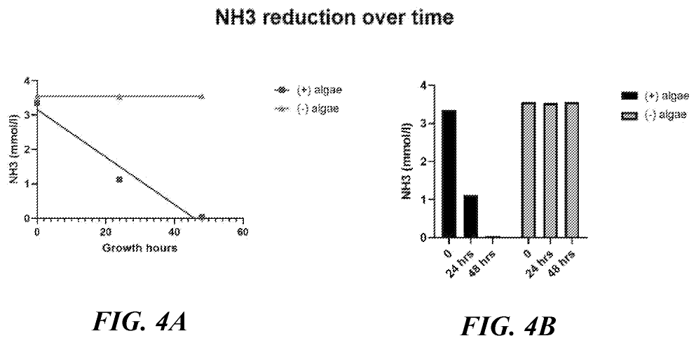

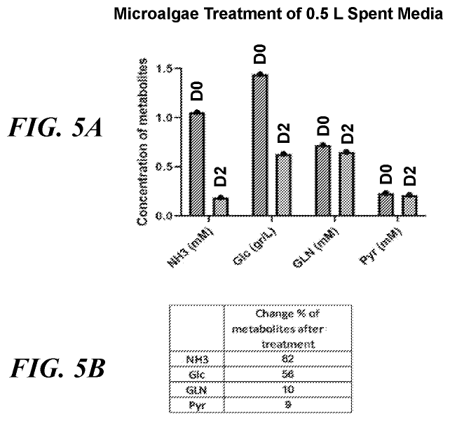

A concentration of 3.5 mM ammonia was reduced in only two days from the spent media by microalgae (FIGS. 4A and 4B). Furthermore, it was determined a volume of 0.5 liters of fish cell spent media was recycled using only 10 ml of Chlorella vulgaris (at a density of 1×10⁸ cells/ml) within a short span of just two days, demonstrating the microalgae's effectiveness in treating large volumes of spent media (FIG. 5A). Specifically, the microalgae efficiently consumed 82% of the ammonia present (FIG. 5B). It was observed that there remained a surplus of sugars, such as glucose and pyruvate, that could be utilized by animal cells at a later stage. Additionally, essential amino acid Glutamine was nearly depleted by the algae during this process.

### Phosphate Reduction

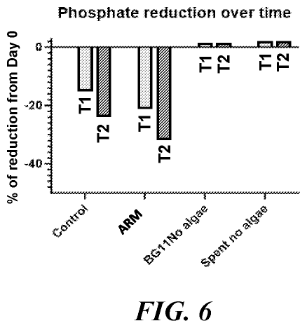

FIG. 6 demonstrates that a 32% reduction in phosphate levels by the microalgae was achieved over two days of cultivation on spent media (“ARM”), whereas the phosphate reduction level on algae reference medium (BG11) was 23% (“Control”). The phosphate was stable in mediums without algae (“BG11No algae” or “Spent no algae”).

### Screening of Additional Microalgae

To assess the potential of other microalgae to thrive in spent media, Porphyridium purpureum, Duneliela salina, and Tetraselmis striata were introduced onto agar plates containing the spent medium. Results indicated that Porphyridium purpureum exhibited growth in the spent medium without requiring osmolarity adjustments. However, Duneliela salina did not thrive compared to the control. Tetraselmis required re-testing.

In sum, the findings of the study demonstrate it is feasible to use microalgae to reduce by-products of cell growth in spent media. Additionally, this study demonstrates the microalgae is effective in reducing ammonia as well as phosphate. This Example highlights the possibility of re-using spent media, after microalgae growth and treatment, for cell culture.

### B. Example 2: Use of Algae Extract for Growth Media Enrichment

This example explores a process of using algae extract to enrich culture media and enhance cell proliferation. This processed in described inside the dashed box in FIG. 1.

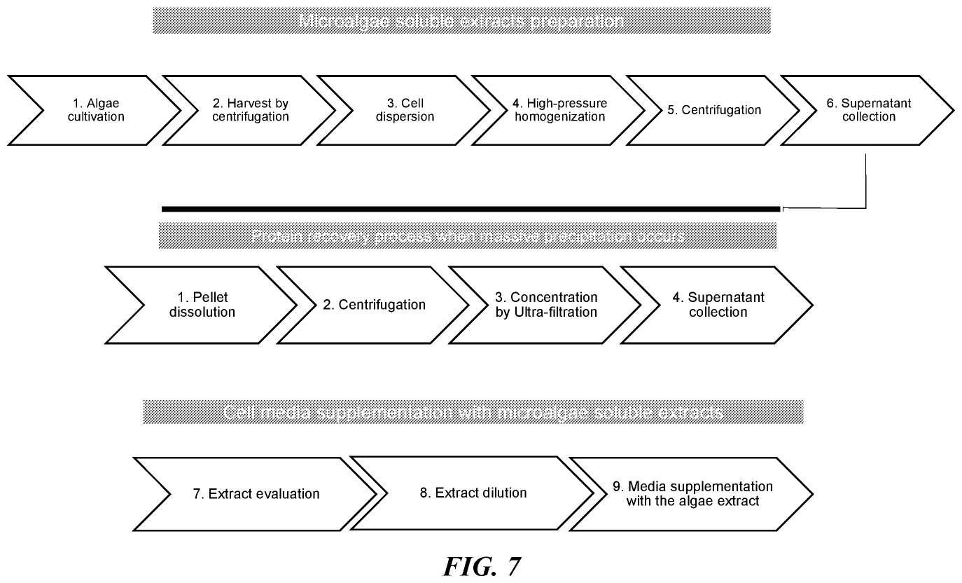

FIG. 7 describes the general process of using microalgae to recycle spent cell culture medium. This process involves extracting nutrients from microalgae using high-pressure homogenization and detergents, centrifugation, and filtration of nutrient extracts. The extract nutrients can be further evaluated, diluted and prepared as a supplement to the recycled media described in Example 1.

Microalgae cultivated in the spent media as described in Example 1 were harvested by centrifugation at 5,000 g and dispersed in a minimal volume of distilled water (DW) or phosphate buffered saline (PBS). The algae cells were lysed in preparation for the extraction of nutrients. To extract algal nutrients, microalgae cells were lysed in isotonic solution or water using high-pressure homogenization at 30-40 kpsi. After lysis by high-pressure homogenization, the resulting lysate was centrifuged at 4° C., at 10,000 g. To continue the protein recovery process, when massive precipitation occurred, the precipitated pellet was treated with phosphate buffered saline (PBS) containing 0.4-1.0% of Triton x-100, for 24 to 72 hours, with shaking at 4° C.

After the detergent treatment, the resulting microalgae mixture was then centrifuged at 4° C. at 10,000 g. The supernatant was collected, filtered through 0.22 μM, and stored at 4° C. To determine the final concentration of the nutrient extract, the OD₂₈₀ was measured. The final concentration, at OD₂₈₀, was >50 mg/ml. Next, the extract was diluted to 10 mg/ml in cell media. The extract was then added to media at 0.01 mg/mL, with or without FBS.

### Cell Proliferation

Microalgae-recycled media was tested for the feasibility of culturing fish cells. To begin, spent media that was treated as described in Example 1 was centrifuged to get rid of the microalgae cells, and then the microalgae recycled medium was filtered through 0.22 μM for sterilization. The microalgae recycled medium was supplemented with nutrients, glucose, glutamine and pyruvate. Fish cells were initially seeded on a fresh medium that was subsequently replaced with varying proportions of the microalgae recycled medium.

Morphology analysis revealed that cells cultured in 50% of the microalgae recycled medium closely resembled that of cells cultured in 100% fresh medium, although the cell density was lower for cells cultured in 50% of the microalgae recycled medium compared to those cultured in fresh medium. There was no significant visual distinction between cells cultured in the microalgae recycled medium, either with or without supplementation after 24 hours of incubation.

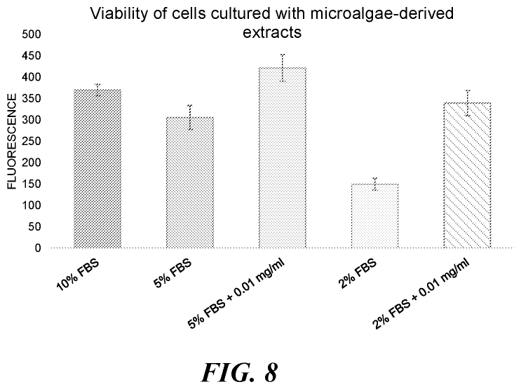

Cells were analyzed using resazurin in a cell viability and proliferation assay. In particular, FIG. 8 demonstrates that cells are viable when cultured in cell growth medium containing microalgae-derived extracts. In both conditions containing a similar amount of fetal bovine serum (FBS), growth conditions including microalgae-derived components enhanced cell proliferation.

In sum, the findings of the study demonstrate it is feasible to use microalgae derived components to supplement cell growth medium. Cells are viable and exhibited enhanced proliferation when the growth media is enriched by microalgae derived components.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1.-73. (canceled)

74. A method of recycling spent media from the cultivation of cells for cultivated food production, comprising:
(a) separating the spent media from the cultured cells;
(b) introducing microalgae to the spent media;
(c) cultivating the microalgae in the spent media under heterotrophic conditions, thus reducing the concentration of ammonia in the media; and
(d) separating the cultivated microalgae from the media to form recycle media,
wherein the recycle media that is separated from the cultivated microalgae is depleted by about 20% to 100% of ammonia.

75. The method of claim 74, wherein the concentration of ammonia is reduced by about 100 percent.

76. The method of claim 74, wherein the microalgae is maintained in a vessel that is held in the dark.

77. The method of claim 74, wherein the recycle media that is separated from the microalgae is depleted by at least 5% of phosphate.

78. The method of claim 74, wherein the culturing is conducted at a temperature ranging from 18-40° C.

79. The method of claim 74, wherein the reduction of ammonia is completed within 1 to 48 hours.

80. The method of claim 79, wherein the method recycles between about 1 to 10 mM ammonia in 48 hours.

81. The method of claim 74, wherein the genus of microalgae in step (b) is Chlorella.

82. The method of claim 81, wherein the species of microalgae in step (b) is Chlorella Vulgaris or Chlorella sp.

83. The method of claim 82, wherein the species of microalgae in step (b) is Chlorella Vulgaris.

84. The method of claim 82, wherein the species of microalgae in step (b) is Chlorella sp.

85. The method of claim 74, wherein the genus of microalgae in step (b) is Phaeodactylum.

86. The method of claim 85, wherein the species of microalgae in step (b) is Phaeodactylum tricornutum.

87. The method of claim 86, wherein the recycled media that is separated from the microalgae is further characterized for being depleted by at least 75% of lactate.

88. The method of claim 87, wherein the reduction of lactate is completed within 1 to 48 hours.
